(19) United States
(12) Patent Application Publication
(10) Pub. No.: US 2025/0235232 A1
(43) Pub. Date: Jul. 24, 2025

(54) CANNULA WITH INTERNAL VISUALIZATION AND TISSUE TARGETING RESECTION DEVICE

(71) Applicant: Mission Surgical Innovations, LLC, Raleigh, NC (US)

(72) Inventor: Brian Garvey, Raleigh, NC (US)

(73) Assignee: MISSION SURGICAL INNOVATIONS, LLC, Raleigh, NC (US)

(21) Appl. No.: 18/800,885

(22) Filed: Aug. 12, 2024

Related U.S. Application Data
(60) Provisional application No. 63/623,667, filed on Jan. 22, 2024.

(51) Int. Cl. A61B 17/32; A61B 1/04; A61B 1/06

(52) U.S. Cl. CPC A61B 17/320016; A61B 1/04; A61B 1/0684; A61B 2017/320052

(57) ABSTRACT

A surgical device including a cannula having a base member at a proximal end, a sidewall connected to the base member, and a bore defined by the base member and sidewall. The cannula includes at least one sensor that may be embedded within any of the surfaces of the cannula, including the inner and outer surfaces of the tip portion, and the inner and outer surfaces of the sidewall extending from the base member. The sidewall of the cannula has an opening into the bore between axially-extending edges of the sidewall. A configurable guide insert includes an opening for insertion of an optical imagining assembly and a surgical instrument. The configurable guide insert may be permanently or detachably coupled to the base member and is positionable in a right orientation for a procedure in a patient's right side or in a left orientation for procedures in a patient's left side.

### CROSS REFERENCE TO RELATED APPLICATION

This application is based upon and claims the benefit of priority from U.S. Provisional Patent Application No. 63/623,667, filed on Jan. 22, 2024, the entire contents of which are incorporated herein by reference.

### FIELD OF INVENTION

The present disclosure relates to a surgical device, and, more particularly, to a surgical device including a cannula including image sensors embedded in the cannula for visualization of the placement of the cannula relative to a target tissue and an internal tissue modification in an action area using a configurable guide insert that may permanently or detachably couple to the cannula.

### BACKGROUND

Conventional soft tissue surgery may be performed endoscopically with the assistance of visualization and tissue manipulation through one of several types of guides or apparatuses. Endoscopic systems for several soft tissue procedures typically utilize insertable cannulas including guides for mounting a scope or camera to the cannula for insertion and manipulation of the camera relative to a target tissue. In addition to mounting a camera to the cannula in such systems, surgical instruments may also be mounted to the cannula. With such endoscopic devices, the ergonomic manipulation of the camera within the cannula in conjunction with the aforementioned surgical instruments may be cumbersome for the surgeon. It would be advantageous to provide a cannula including embedded image sensors within the cannula that accomplishes similar objectives as a mountable camera. Surgeons would then have an enhanced surgical tool with improved ergonomics and visualization, which provides the patient with greater potential for positive outcomes from surgery. It would also be advantageous to provide surgeons with an ability to place an endoscopic device at an incision point and advance or retract one or both of an optical imaging assembly with one or more image sensors and a surgical instrument along an axis of the cannula when both the one or more image sensors and the surgical instrument are present at the incision point.

### SUMMARY

In one aspect, the present disclosure is directed to a cannula for use as part of a surgical device. The cannula may include a housing member within the cannula, with the housing member having an upper surface extending axially along a length of the cannula. The cannula may also include at least one embedded sensor disposed within the housing member, a processor and a memory communicatively coupled to the at least one embedded sensor, a base member at a proximal end of the cannula, a sidewall connected to the base member and extending in an axial direction from the proximal end to a distal end of the cannula, a bore extending in the axial direction defined by each of the upper surface of the housing member and the base member and the sidewall, and an opening extending axially through the base member and into the bore between axially-extending edges of the sidewall. The base member may be configured to slidably receive at least one surgical implement through the opening and into the bore, and the at least one embedded sensor may detect an image data of a target tissue and an adjacent tissue. The target tissue may be manipulated by the at least one surgical implement. The at least one embedded sensor may be an image sensor configured to transmit the image data to a display, and the image sensor may comprise an adjacent region in the upper surface for mounting one or more embedded LEDs within the housing member. Each of the embedded LEDs may be flush with the upper surface. The at least one sensor may comprise a plurality of image sensors configured to transmit a respective image data to a display for visualization of the respective image data of each of the plurality of image sensors, and each of the plurality of image sensors may comprise an adjacent region in the upper surface for mounting one or more embedded LEDs within the housing member. The housing member including the plurality of image sensors and the one or more embedded LEDs may comprise an assembly, and the assembly may be configured to slide along the length of the cannula between the axially-extending edges of the sidewall in the axial direction between the proximal end and the distal end of the cannula. The base member may be configured to slidably receive the assembly through the opening and between the axially-extending edges of the sidewall. The assembly may further comprise at least one fixed position engagement portion and the cannula may comprise at least one fixed position engagement point. The at least one fixed engagement portion may be configured to matingly couple with the at least one fixed position engagement point and may provide a selectable incremental positioning of the assembly within the cannula. The base member may include a configurable guide insert detachably coupled to the base member, and the configurable guide insert may include a first opening extending axially through the base member into an action area outside the opening into the bore and radially between edges of the sidewall. The configurable guide insert may further include a second opening adjacent to the first opening, and the second opening may extend axially through the base member into the bore. The base member may be configured to slidably receive the housing member through the second opening and into the bore. The configurable guide insert may be detachably coupled to be positionable in a configurable orientation comprising a right orientation for positioning of the surgical implement for a surgical procedure in a right side of a patient, or a left orientation for positioning of the surgical implement for the surgical procedure in a left side of the patient. In addition, the configurable guide insert may be configured for rotation of 180 degrees about an axis defined in relation to the cannula when positioning the configurable guide insert on the base member in the right orientation or in the left orientation.

In another aspect, the present disclosure is directed to a tissue visualization system. The tissue visualization system may comprise a cannula having a proximal and a distal end. The cannula may include a housing member within a cannula, with the housing member having an upper surface extending axially along a length of the cannula. The tissue visualization system may further include at least one embedded sensor disposed within the housing member, a processor and a memory communicatively coupled to the at least one embedded sensor, a base member at a proximal end of the cannula, a sidewall connected to the base member and extending in an axial direction from the proximal end to a distal end of the cannula, a bore extending in the axial direction defined by each of the upper surface of the housing member and the base member and the sidewall, and an opening extending axially through the base member and into the bore between axially-extending edges of the sidewall. The base member may be configured to slidably receive at least one surgical implement through the opening and into the bore, and the at least one embedded sensor may detect an image data of a target tissue and an adjacent tissue, with the target tissue to be manipulated by the at least one surgical implement. The at least one embedded sensor may be an image sensor configured to transmit the image data to a display, and the image sensor may comprise an adjacent region in the upper surface for mounting one or more embedded LEDs within the housing member, with each of the embedded LEDs flush with the upper surface. The at least one sensor may comprise a plurality of image sensors configured to transmit a respective image data to a display for visualization of the respective image data of each of the plurality of image sensors, and each of the plurality of image sensors may comprise an adjacent region in the upper surface for mounting one or more embedded LEDs within the housing member. The housing member including the plurality of image sensors and the one or more embedded LEDs may comprise an assembly, and the assembly may be configured to slide along the length of the cannula between the axially-extending edges of the sidewall in the axial direction between the proximal end and the distal end of the cannula. The base member may be configured to slidably receive the assembly through the opening and between the axially-extending edges of the sidewall. The assembly may further comprise at least one fixed position engagement portion and the cannula may comprise at least one fixed position engagement point. The at least one fixed engagement portion may be configured to matingly couple with the at least one fixed position engagement point and may provide a selectable incremental positioning of the assembly within the cannula. The base member may include a configurable guide insert detachably coupled to the base member, and the configurable guide insert may include a first opening extending axially through the base member into an action area outside the opening into the bore and radially between edges of the sidewall. The configurable guide insert may further include a second opening adjacent to the first opening, and the second opening may extend axially through the base member into the bore. The base member may be configured to slidably receive the housing member through the second opening and into the bore. The configurable guide insert may be detachably coupled to be positionable in a configurable orientation comprising a right orientation for positioning of the surgical implement for a surgical procedure in a right side of a patient, or a left orientation for positioning of the surgical implement for the surgical procedure in a left side of the patient. In addition, the configurable guide insert may be configured for rotation of 180 degrees about an axis defined in relation to the cannula when positioning the configurable guide insert on the base member in the right orientation or in the left orientation.

In another aspect, the present disclosure is directed to a surgical device. The surgical device may comprise a cannula having a proximal and a distal end. The cannula may include a housing member within a cannula, with the housing member having an upper surface extending axially along a length of the cannula. The surgical device may further include at least one embedded sensor disposed within the housing member, a processor and a memory communicatively coupled to the at least one embedded sensor, a base member at a proximal end of the cannula, a sidewall connected to the base member and extending in an axial direction from the proximal end to a distal end of the cannula, a bore extending in the axial direction defined by each of the upper surface of the housing member and the base member and the sidewall, and an opening extending axially through the base member and into the bore between axially-extending edges of the sidewall. The base member may be configured to slidably receive at least one surgical implement through the opening and into the bore, and the at least one embedded sensor may detect an image data of a target tissue and an adjacent tissue, with the target tissue to be manipulated by the at least one surgical implement. The at least one embedded sensor may be an image sensor configured to transmit the image data to a display, and the image sensor may comprise an adjacent region in the upper surface for mounting one or more embedded LEDs within the housing member, with each of the embedded LEDs flush with the upper surface. The at least one sensor may comprise a plurality of image sensors configured to transmit a respective image data to a display for visualization of the respective image data of each of the plurality of image sensors, and each of the plurality of image sensors may comprise an adjacent region in the upper surface for mounting one or more embedded LEDs within the housing member. The housing member including the plurality of image sensors and the one or more embedded LEDs may comprise an assembly, and the assembly may be configured to slide along the length of the cannula between the axially-extending edges of the sidewall in the axial direction between the proximal end and the distal end of the cannula. The base member may be configured to slidably receive the assembly through the opening and between the axially-extending edges of the sidewall. The assembly may further comprise at least one fixed position engagement portion and the cannula may comprise at least one fixed position engagement point. The at least one fixed engagement portion may be configured to matingly couple with the at least one fixed position engagement point and may provide a selectable incremental positioning of the assembly within the cannula. The base member may include a configurable guide insert detachably coupled to the base member, and the configurable guide insert may include a first opening extending axially through the base member into an action area outside the opening into the bore and radially between edges of the sidewall. The configurable guide insert may further include a second opening adjacent to the first opening, and the second opening may extend axially through the base member into the bore. The base member may be configured to slidably receive the housing member through the second opening and into the bore. The configurable guide insert may be detachably coupled to be positionable in a configurable orientation comprising a right orientation for positioning of the surgical implement for a surgical procedure in a right side of a patient, or a left orientation for positioning of the surgical implement for the surgical procedure in a left side of the patient. In addition, the configurable guide insert may be configured for rotation of 180 degrees about an axis defined in relation to the cannula when positioning the configurable guide insert on the base member in the right orientation or in the left orientation.

### BRIEF DESCRIPTION OF THE DRAWINGS

The foregoing summary and the following detailed description will be better understood when read in conjunction with the appended drawings, which illustrate a preferred embodiment of the invention. In the drawings:

FIG. 1A is an exploded view of a tissue visualization system including a cannula, an optical imaging component, and a surgical instrument.

FIG. 1B is a perspective view of a tissue visualization system in which the optical imaging component is fixed within the cannula.

FIG. 2A is a perspective view of a configurable guide insert for use as part of the surgical device;

FIG. 2B is a perspective view of a cannula with generic opening for use as part of the surgical device;

FIG. 2C is a perspective view of an obturator for use as part of the surgical device;

FIG. 2D is a side-perspective view of the surgical device with all components in place;

FIG. 3A is a perspective view of a configurable guide insert lockingly engaging within the base member of a cannula;

FIG. 3B is a perspective view of a configurable guide insert that is wholly contained by and attached to the base member of a cannula;

FIG. 4A is an enlarged perspective view of the proximal portion of a right orientation of the configurable guide insert;

FIG. 4B is an enlarged perspective view of the top portion of a right orientation of the configurable guide insert;

FIG. 5A is an enlarged perspective view of the proximal portion of a left orientation of the configurable guide insert;

FIG. 5B is an enlarged perspective view of the top portion of a left orientation of the configurable guide insert;

FIG. 6A is a top perspective view of a left orientation of a configurable guide insert lockingly engaging within the base member of a cannula;

FIG. 6B is a proximal perspective view of a left orientation of a configurable guide insert lockingly engaging within the base member of a cannula;

FIG. 6C is a proximal perspective view of a left orientation of both an obturator and a configurable guide insert within the base member of a cannula;

FIG. 6D is a perspective view of a patient's left hand showing an incision point for a left orientation of the surgical device;

FIG. 7A is a top perspective view of a right orientation of a configurable guide insert lockingly engaging within the base member of a cannula;

FIG. 7B is a proximal perspective view of a right orientation of a configurable guide insert lockingly engaging within the base member of a cannula;

FIG. 7C is a proximal perspective view of a right orientation of both an obturator and a configurable guide insert within the base member of a cannula;

FIG. 7D is a perspective view of a patient's right hand showing an incision point for a right orientation of the surgical device;

FIG. 8A is a side view of an assembly housing including one or more image sensors and additional optical imaging components within an exemplary surgical device;

FIG. 8B is a side view of a rasp within an exemplary surgical device;

FIG. 8C is a side view of a reverse cutting knife within an exemplary surgical device;

FIG. 8D is a side view of a forward cutting knife within an exemplary surgical device; and

FIG. 9 is a block diagram of an example device in which one or more disclosed embodiments can be implemented.

### DETAILED DESCRIPTION OF THE PREFERRED EMBODIMENT(S)

Various embodiments of the invention are described in the following paragraphs. Where like elements have been depicted in multiple embodiments, identical or similar reference numerals have been used for ease of understanding. Also, it is to be understood that the disclosed embodiments are merely examples, and other embodiments can take various and alternative forms. The figures are not necessarily to scale; some features could be exaggerated or minimized to show details of particular components. Therefore, specific structural and functional details disclosed herein are not to be interpreted as limiting but merely as a representative basis for teaching one skilled in the art to employ various embodiments. As those of ordinary skill in the art will understand, various features illustrated and described concerning any one of the figures can be combined with features illustrated in one or more other figures to produce embodiments that are not explicitly illustrated or described. The combinations of features illustrated provide representative embodiments for typical applications. However, various combinations and modifications of the features consistent with the teachings of this disclosure could be desired for particular applications or implementations.

The terminology used herein is to describe particular aspects only and is not intended to limit the scope of the present disclosure. Unless defined otherwise, all technical and scientific terms used herein have the same meaning as commonly understood to one of ordinary skill in the art to which this disclosure belongs. Although any methods, devices, or materials similar or equivalent to those described herein can be used in the practice or testing of the disclosure, the following example methods, devices, and materials are now described.

Several soft tissue procedures involve the division or release of a soft tissue (ligament, tendon, muscle, fascia, etc.) with a knife blade to decompress adjacent nerves or other soft tissues. Conventional soft tissue surgery is performed either in an open manner with large incisions; in a mini-open manner with a smaller incision, or endoscopically with the assistance of visualization and tissue manipulation through one of several types of guides or apparatuses. Open procedures are more disruptive to the patient and often do not utilize surgical guides to protect the adjacent soft tissue structures. Open and mini-open procedures require the incision to be placed directly over the structure subject to release. Often times the incision in an open or mini-open procedure must be made in an anatomic location that provides additional discomfort to the patient and increased potential for post-operative complications. Endoscopic systems comprise guides that allow the incision placement adjacent to the tissue being released. Surgeons may then have more variability in the placement of the incision which provides the patient with a potential for faster recovery and decreased pain or discomfort post-surgery.

In general, conventional endoscopic systems have several limitations depending on their independent design features. Many conventional endoscopic systems do not allow the blade to move independently from the endoscope or arthroscope. These systems may require the scope or camera to be mounted to the blade mechanism, which may be static or deployed through a secondary mechanism. The inability to move the blade independently from the camera through a guide or cannula limits the physical control and visualization available to the surgeon. The surgeon's ergonomic manipulation of the blade and ability to see the target tissue may be compromised by the camera. This can prevent the surgeon from confirming with 100% certainty that the soft tissue was appropriately manipulated or released. Additionally, the inability to move the blade independently from the camera provides the potential for the surgeon to cut adjacent soft tissues, such as nerves, without knowing. Also if the view through the camera becomes obscured via moisture or debris, the surgeon must remove the entire apparatus from the body, disassemble the camera from the guide and blade, remove the obstruction to the view, reassemble and reinsert the apparatus. This practice adds significant time and inconvenience to the surgery. Accordingly, it may be advantageous to provide a cannula including embedded image sensors within the cannula that accomplishes similar objectives as a mountable camera to achieve improved ergonomics and visualization of the manipulation of a target tissue.

Further, in the area of endoscopic systems for soft tissue release, the position of the surgical implement, such as the blade, relative to the one or more image sensors, may become significant. For example, the physician may need to insert an endoscopic system at a narrow incision point. Depending on the preference of the physician, consideration may be made regarding whether the one or more image sensors is positioned below the surgical implement—in a top-to-bottom configuration—or if the one or more image sensors are positioned on a side of the surgical implement—in a side-to-side configuration. The efficiency of insertion and placement of the device at the incision point may be taken into account by the physician relative to a preferred configuration of the device, for insertion at a narrow incision point. For example, a physician's ability to visualize the target tissue during a procedure may depend on whether the device is configured with a top-to-bottom configuration or a side-to-side configuration. Accordingly, there is a need for an endoscopic soft tissue release device that provides improved functionality for insertion and placement of the device and cannula, while also providing improved visibility of the surgical implements during the procedure. It may be advantageous to implement an endoscopic soft tissue release device that features one or more surgical implements in a position that is horizontally adjacent to the one or more image sensors. It may also be advantageous to implement an endoscopic soft tissue release device that features one or more surgical implements that is above the one or more image sensors, thereby providing improved functionality and optionality for insertion and placement of the device at narrow incision points and improved visibility of the implements during surgery.

In addition, the location of a surgical incision point becomes significant in relation to the physician's objective to operate on targeted soft tissue structures for the division or release of a soft tissue (ligament, tendon, muscle, fascia, etc.). In addition to the targeted soft tissue structures, the physician must also account for the non-targeted, adjacent soft tissue structures. Whether a surgical incision point resides on the left or right side of a particular anatomical structure, such as the hand, involves additional consideration for avoiding contact of a surgical implement, such as a cutting instrument, with the adjacent soft tissue structures of the left or right hand during a surgical procedure. For example, when an incision point resides at the ulnar side of a patient's left hand, the physician must remain away from non-targeted soft tissue structures when manipulating a cutting instrument. Accordingly, it would also be advantageous if the physician were able to configure an endoscopic soft tissue release device at the time of surgery based upon where the incision will be made at a particular point of an anatomical structure, such as the ulnar side of a patient's left or right hand. The ability to position such an endoscopic soft tissue release device in the optimum orientation with respect to the location of the incision and surrounding soft tissue structures would provide for a more efficient and ergonomic surgical device.

In addition, the orientation of an optical sensor and a cutting instrument within a cannula that is utilized for division or release of a soft tissue, may also be considered in relation to whether the physician is left or right handed. For example, when performing a soft tissue release procedure on the ulnar side of the wrist, the ability of the physician to perform each surgical maneuver ergonomically in preference to the physician's dominant hand may also be a significant factor. Accordingly, there is a need for an improved endoscopic soft tissue release device that enables the physician to configure the surgical instrument to achieve ergonomic advantages relative to the location of the incision point and the dominant hand of the physician.

The present disclosure is directed to overcoming one or more problems of the prior art.

As described in the embodiments herein, the present disclosure provides an improved surgical device including image sensors which is generally applicable to procedures that include the internal visualization of insertion of an implement through an incision and the manipulation of a selected tissue with at least one implement. The disclosed surgical device includes multiple components, which are used in conjunction with each other to execute the internal visualization and tissue targeting and resection and which include features that enhance and ease such execution.

As described in the embodiments herein, the image sensors (optical sensors) may be disposed or embedded within the surfaces of the cannula, including the inner and outer surfaces of the tip portion of the cannula, and the inner and outer surfaces of the planes extending from the base member of the cannula. The image sensor may provide a relay of information regarding the action area located outside of the bore and radially between the edges of the sidewall.

The image sensors in the canula may provide for the relaying of information along the length of the cannula about the location, existence, or condition of target tissue or any adjacent tissue. The sensors may also relay information regarding an action area for internal tissue modification, in a singular resection or excision, in situ. One or more or all of the image sensors of the surgical device may include capability for heat, infrared, and ultrasonic sensing and measurement. For example, one or more of the image sensors may be configured for echo detection and measurement as well as sonar, lidar and radar detection. In addition, the sensors may feature a small form factor that may be square or rounded shape. In an embodiment, the sensors may have a small form factor of approximately 1 millimeter in length, or 1 square millimeter, so that it may conveniently accompany the size and dimensions of the cannula and the housing of the optical imaging components.

In addition, the one or more image sensors may be configured as an image sensor or a camera located on a chip that may be embedded in a surface of the cannula. The chips may feature a small form factor that may be a square or rounded shape. In an embodiment, a chip may have a small form factor of approximately 1 millimeter in length, or 1 square millimeter, so that it may conveniently accompany the size of the cannula planes in which one or more of the chips may be embedded.

When embedded on an inner or outer plane of the cannula, a chip with image sensor may be flush with the plane of the cannula. For example, if one or more of the chips with image sensors is located on the inner surfaces of the tip portion of the cannula and on the inner surfaces of the planes extending from the base member, the chips may be flush with these planes so that the chips do not protrude outwardly from the planes of the cannula. When such chips are embedded and flush with the inner planes of the cannula, the surgeon may slidably move a surgical instrument across a bottom surface of the canula that includes the embedded image sensors, so that the image sensors do not interfere with the smooth movement of the surgical instrument along the bottom surface of the cannula. The inner planes of the cannula which may be touched or in contact with a surgical instrument include the inner planes of the axially-extending edges of the sidewall of the cannula as well as the inner plane running parallel and tangential to the axially-extending edges of the sidewall.

In other embodiments, a chip with image sensor may be sub-flush with the plane of the cannula. In particular, when a chip with image sensor is sub-flush with a plane of the cannula, there might be a plastic layer or plastic portion between the image sensor and the outermost plane surface of the cannula that is exposed to the surgical instruments that may slidably move across a surface of the cannula. For example, if one or more of the chips with image sensors is located on the inner surfaces of the tip portion of the cannula and on the inner surfaces of the planes extending from the base member, the chips may be sub-flush with these planes so that the chips may be embedded within a cannula surface and do not protrude outwardly from the planes of the cannula surface. When such chips are embedded and sub-flush with the inner planes of the cannula, the surgeon may slidably move a surgical instrument across a bottom surface of the canula that includes the embedded image sensors, so that the image sensors do not interfere with the smooth movement of the surgical instrument along the bottom surface of the cannula. The inner planes of the cannula which may be touched or in contact with a surgical instrument include the inner planes of the axially-extending edges of the sidewall of the cannula as well as the inner plane running parallel and tangential to the axially-extending edges of the sidewall.

In other embodiments, a chip with image sensor may protrude through the plane of the cannula. In particular, when a chip with image sensor protrudes through a plane of the cannula, the chip with image sensor may be exposed to the surgical instruments that may slidably move across a surface of the cannula and adjacent to the chip with image sensor protruding through the plane of the cannula. When the chips may protrude outwardly from the planes of the cannula surface, the surgeon may slidably move a surgical instrument across a bottom surface of the canula and adjacent to the protruding image sensors, so that the image sensors may protrude in a position adjacent to the smooth movement of the surgical instrument along the bottom surface of the cannula. When a chip with image sensor protrudes through a plane of the cannula, the chip with image sensor may be exposed to the bore of the cannula.

The surgical instruments as described herein that may be used in conjunction with the surgical device including a cannula with image sensors may provide for probing, cutting, injecting, excising or otherwise examining or targeting at least one tissue for the subject of surgical intervention or diagnostic analysis. The instruments may be placed into an action area of the cannula to manipulate, probe a tissue structure, while under the monitoring of the embedded sensors.

The image sensors on the cannula may located and spaced anywhere inside or outside of the cannula to deliver a clear view of a target and adjacent tissues. The image sensor may also be disposed within, adjacent to, or in or about an action area of the cannula. In an embodiment, the image sensors may comprise a selected portion of an action area of the cannula surface where a physician may adjacently access a target tissue for operation or resection. The image sensors may be arranged in a defined array of image sensors, such as in a cluster or pattern about the surfaces of the cannula. A position of the image sensors, including an array of image sensors, may or may not be adjustable relative to a patient's anatomy.

In an embodiment, the image sensors disposed on the surfaces of the cannula may encompass a length of the cannula or a portion of a length of the cannula to allow visualization of the entire segment of the anatomy in which the surgical device is inserted within a patient.

In another embodiment, the image sensors may each take multiple images, so that such images may further be compiled together to form one larger image.

In another embodiment, a cannula including multiple image sensors may display either a continuous single image or combination of images from various image sensors and vantages points.

As understood by those of skill in the art, the image sensors may be used in conjunction with external computer and processing hardware and software to process, store and analyze the data and images that are captured, measured and detected by the image sensors in the cannula. For example, the image sensors may be wirelessly connected to a screen for visualization. The image sensors and chips as described herein may include conventional components (e.g., camera components, wireless electronic components, wireless connector components) which allow an captured images and data to be presented to a physician performing the procedure (e.g., via a connected display). In addition, the image sensors may connect to an external output via cable or an over the air transmission. Further, the sensors may be interchangeable, reconfigurable, disposable, or otherwise consumable within a procedure and replaced for subsequent procedures.

The image sensors as described herein may also be part of an assembly including one or more image sensors (optical sensors), and additional optical imaging components including a light source such as an LED, a flexible circuitry for carrying the captured image(s) from the image sensor to a coupler, an optical grade protective housing covering the image sensor and the light source, a rigid or semi-rigid conduit for enclosing the flexible circuitry, a coupler for connection to an optical display, and a connection mechanism for optical image transfer to another device. It is to be understood that in embodiments of the surgical device including an ability to wirelessly transmit image data from the image sensors to a display, the connection mechanism for optical image transfer to another device may not be necessary.

In an embodiment, the assembly for the one or more image sensors may be permanently fixed to a lower portion of the cannula.

In another embodiment, the assembly for the one or more image sensors may be entirely removably from the cannula.

In yet another embodiment, the assembly for the one or more image sensors may be permanently fixed to a lower portion of the cannula while also having additional capability for slidable movement and adjustable positioning within the cannula along a longitudinal axis of the cannula.

In an embodiment, the disclosed surgical device may include a cannula which forms a base structure that may include one contiguous opening with space to accommodate insertion of at least one surgical instrument in addition to the assembly for the image sensors. The at least one surgical instrument may be restricted to only stay within a respective designated region within the singular contiguous opening through the base structure. More specifically, the singular contiguous opening through the base structure may include one or more defined openings, which define an axially-extending space for receiving other components of the surgical device, including the at least one surgical instrument and the assembly for the image sensors. The opening defines the designated region within the singular contiguous opening through the base member for the restricted positioning and movement of the at least one surgical instrument and the assembly for the image sensors.

In another embodiment, the disclosed surgical device may include a cannula, which forms a base structure, a configurable guide insert that detachably couples to the cannula and that may include a combination track defined along an axis of the cannula, and an obturator that may be inserted through the combination track of the configurable guide insert and into the cannula along the axis of the cannula. The combination track of the configurable guide insert may include one contiguous opening with space to accommodate insertion of one or more surgical instruments, including the at least one surgical instrument and the assembly for the image sensors. The one or more surgical instruments and assembly for the image sensors may be restricted to only stay within their respective designated regions within the singular contiguous opening through the combination track of the configurable guide insert. More specifically, the singular contiguous opening through the combination track of the configurable guide insert may include one or more openings, which define an axially-extending space for receiving other components of the surgical device. The opening defines the designated region within the singular contiguous opening through the configurable guide insert for the restricted positioning and movement of the at least one surgical instrument and the assembly for the image sensors within the configurable guide insert.

For example, in an embodiment, the singular contiguous opening through the combination track of the configurable guide insert may include one or more openings, which may be at least a first slot opening and a second slot opening. The first slot opening may receive a first surgical implement and the second slot opening may receive an assembly including one or more image sensors. The first surgical implement may be a first tissue manipulation device such as a first cutting or resection tool, which is guided by the physician performing the procedure and which may be used to manipulate (e.g., cut) the target tissue. In particular, a tissue manipulation device may be any of a rasp, a forward cutting knife, a reverse cutting knife, or any other surgical implement, for example.

The disclosed cannula having a configurable guide insert with combination track provides an improved mechanism for performing a tissue manipulation procedure. In one embodiment, the one or more openings including at least the first slot opening and the second slot opening of the combination track, which may be interconnected in space, allow different surgical implements to be positioned in close proximity to the sensors for visualization of a target tissue, providing for effective interaction and use of one or more implements and the image sensors. The combination track of the configurable guide insert provides a guide structure for the at least one surgical implement relative to the sensors.

In addition, the configurable guide insert may provide enhanced functionality by enabling the physician to select between the two configurations for the device during a surgical procedure. The configurable guide insert may be detachably coupled to the base member of the cannula in a selected orientation with respect to the incision point of an anatomical structure, such as the ulnar side of a patient's left or right hand. Markings on the configurable guide insert may indicate the respective orientation of the device, so that the physician may quickly assemble the device by detachably coupling the configurable guide insert to the cannula, for a rigid attachment of the cannula, configurable guide insert and obturator components, in the optimum orientation, for insertion of the device at a designated incision point in a left side or right side of a patient with precise target tissue visualization via the sensors in the assembly

In addition to the ability to assemble the device in a desired configuration, the device also provides an additional ergonomic advantage based on the image sensors residing within and being embedded within the cannula, as opposed to inserting a camera instrument or scope into the surgical device, in addition to the surgical instruments. For example, current endoscopic systems for soft tissue release are difficult for the physician to insert and place at the incision point because the position of the surgical implement, such as the cutting tool, is oriented below the insertable camera device or scope, in a top-to-bottom relationship. With the current systems, when the cannula features a cutting tool above the camera or scope, the height of the cannula is greater, making it more cumbersome to insert the cannula at a narrow incision point. Moreover, the physician's visibility of the cutting tool via the camera device may be compromised when the camera is below the cutting tool. Therefore, a cannula featuring embedded image sensors within the cannula, as described herein, simplify insertion of the cannula at the narrow incision point while also improving the physician's visibility of the surgical implements via the embedded image sensor.

In addition, the image sensors and the assembly including the additional optical imaging components as described herein may be recyclable or disposable or reusable. The surgical device, including products or kits, or components of the surgical device, may be reusable, disposable, recyclable. It is to be understood that in various embodiments, all of the components of the surgical device or none of the components of the surgical device are kitted (sterile). In other embodiments, the surgical device may provide for some other combination of sterile or non-sterile aspects of a system for internal soft tissue resection.

The disclosed surgical device may be provided to the physician at the time of surgery in a single kit that includes the cannula, the assembly including the one or more image sensors (optical sensors), the additional optical imaging components, the configurable guide insert, and the obturator. The kit may be a sterile packaged kit for surgery that may be assembled at the time of an operation with additional functionality to configure the device in the desired orientation for targeting of soft tissue in a patient's left or right side, such as a left or right hand, for example.

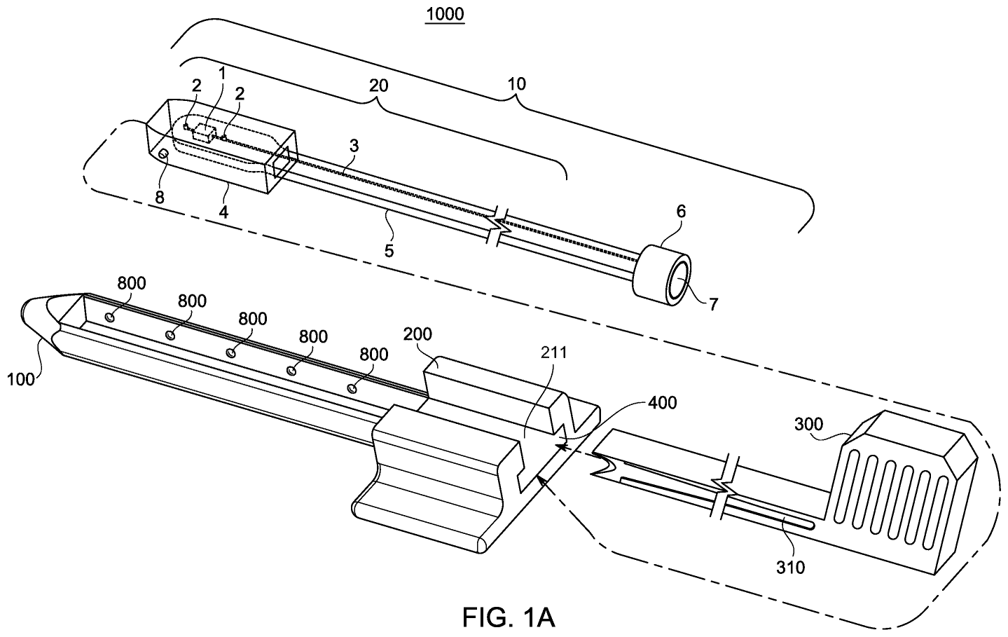

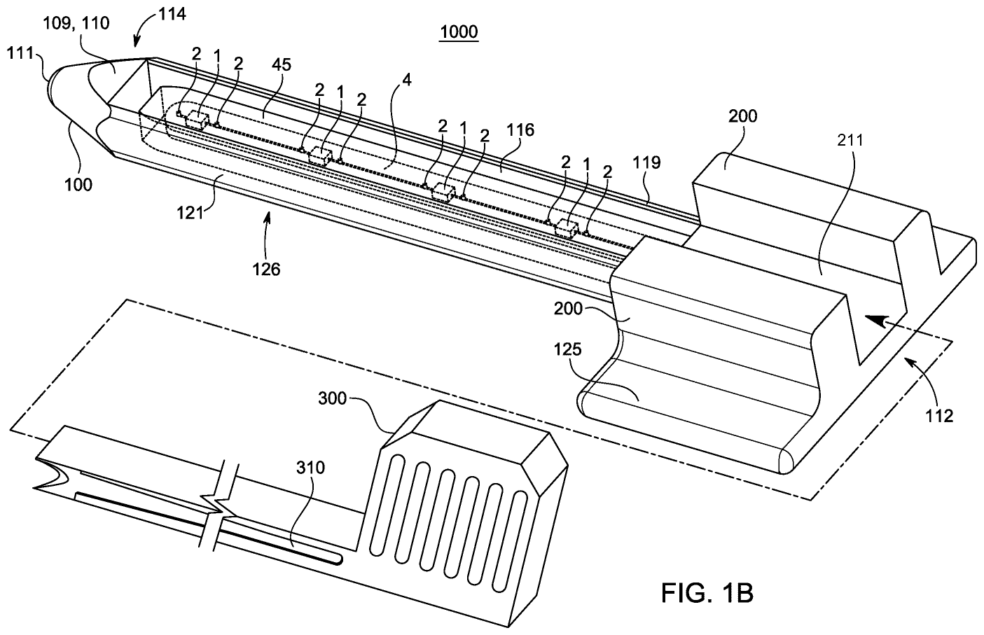

FIG. 1A is an exploded view of a tissue visualization system and surgical device 1000, including a cannula 100, an assembly 10, including the image sensor(s) (optical sensor(s) 1, and additional optical imaging components 2, 3, 4, 5, 6, 7, 8, and a surgical instrument 300. The additional optical imaging components may include a light source 2 such as an LED, a flexible circuitry 3 for carrying the captured image(s) from the image sensor to a coupler 6, an optical grade protective housing 4 covering the image sensor 1 and the light source 2, a rigid or semi-rigid conduit 5 for enclosing the flexible circuitry 3, a coupler 6 for connection to an optical display (not shown in FIG. 1A), and a connection mechanism 7 for optical image transfer to another device.

The surgical device 1000 is configured to be used in a surgical procedure, such as a tissue manipulation procedure. For example, the surgical device 1000 may be configured to be used in a procedure for releasing, excising, or modifying soft tissue structures. Examples of such procedures, which may be carried out using the surgical device 1000 include carpal tunnel release, cubital tunnel release, trigger finger release, gastrocnemius release, and plantar facia release. These and similar procedures normally include minimally-invasive techniques, which may be generally assisted through the use of one or more image sensors 1 that provide visualization of an internal structure, which is being targeted for the procedure. The surgical device 1000 includes features, which allow for such a visualization device to be used in combination with at least one surgical instrument 300, used to carry out the surgical component of the procedure.

The one or more image sensors 1 as described herein may enable the physician to accurately visualize tissue structures adjacent to the cannula 100 both when inserting the cannula 100 within an incision point and when navigating and placing the cannula 100 near a target tissue. A target tissue, as described herein, may be a tissue that the physician manipulates with one or more surgical instruments 300 that may be used in combination with the cannula 100. The one or more image sensors 1 may enable the physician to track a position and orientation of the cannula 100 relative to adjacent tissue and target tissue. In addition, the one or more sensors 1 may detect, capture, gather, and transmit optical image data to a display screen to enable the physician to achieve confirmation that the target tissue may be manipulated without engaging adjacent tissues. When deploying a cannula 100 with multiple sensors 1, image data from each of the sensors 1 may be transmitted to one or more display screens and viewed by the physician at the same time in a multi-view display. Although the one or more sensors 1 as described herein have been described as optical image sensors, it is understood that the one or more sensors 1 in other embodiments may alternatively be provided and configured as other types of sensors. For example, sensors in the device 1000, as disclosed herein, may alternatively and also include a gyroscope to measure orientation in space and angular velocity. In an embodiment, the surgical device 1000 may be configured to provide for simultaneous use of multiple surgical instruments at one time with visualization provided by an assembly 10 including a housing 4 encasing one or more image sensors 1. The first surgical instrument 300 may be a cutting tool or scalpel, and a second surgical implement (not shown in FIG. 1A) may be a tissue manipulation device, such as a rasp, a probe, a hook, a feeler, a reverse or antegrade cutting implement, a forward cutting implement, or the like. When a physician inserts the cannula 100 including the assembly 10 at an incision point to access a target tissue, the cannula 100 may provide a passageway for the first surgical instrument 300 to manipulate a target tissue without manipulating tissue structures adjacent to the target tissue. The one or more sensors 1 that may be optical image sensors may transmit image data to one or more display screens or monitors to allow for confirmation by the physician that the target tissue is to be manipulated without engaging adjacent tissues. In an embodiment, the cannula 100 may guide the movement of a first surgical instrument 300 and also a second surgical instrument for manipulation of the target tissue. When the physician deploys an embodiment of the cannula 100 featuring multiple sensors 1, their image data may each be transmitted to a single display screen and viewed at the same time to provide a multi-view capability of the data from each of the sensors 1.

An assembly 10 including one or more image sensors (optical sensors) 1, and the additional optical imaging components 2, 3, 4, 5, 6, 7, 8, which allow an image to be presented to a physician performing the procedure (e.g., via a connected display) may be permanently fixed within the cannula 100. In other embodiments of the surgical device 1000, the assembly 10 for the one or more image sensors 1 and the additional optical imaging components 2, 3, 4, 5, 6, 7, 8 may be entirely removably from the cannula 100. In yet another embodiment of the surgical device 1000, the assembly 10 for one or more image sensors 1 and the additional optical imaging components 2, 3, 4, 5, 6, 7, 8 may be permanently fixed to a lower portion of the cannula 100 while also having additional capability for slidable movement and adjustable positioning within the cannula 100 along a longitudinal axis of the cannula 100.

These and other objects, features and advantages of the surgical device 1000 will be described in further detail in the following illustrative embodiments thereof.

Referring again to FIG. 1A, the surgical device 1000 may include a cannula 100, having a proximal end 112 and a distal end 114 along an axial direction of the cannula 100, and a bore 116 extending along the axial direction through the cannula 100. The cannula 100 includes a base member 200 at the proximal end 112, an end portion 109 at the distal end 114, and a tubular body portion 126 formed there between. The end portion 109 may include a support surface 110. The bore 116 may extend an entire length of the cannula 100. In an embodiment, the end portion 109 is closed to act as a stop for a surgical implement 300, such as a cutting instrument or scalpel.

Referring again to FIG. 1A, the tubular body portion 126 forms a surface for supporting and guiding at least one surgical implement 300, such as a cutting instrument or scalpel, in some manner. The tubular body portion 126 extends axially from the base member 200 to the support surface 110. The tubular body portion 126 includes a sidewall 119, which extends radially to form a supporting member for at least one surgical implement 300, which may be the cutting implement, for example. The sidewall 119 extends axially and radially and defines the bore 116 therethrough and includes a pair of axially-extending edges 121, which define an opening into the bore 116 (e.g., from above). In an exemplary embodiment, the area between the edges 121 of the sidewall 119 is also above the opening into the bore 116 when the cannula 100 is inserted into a patient through an incision (i.e., during use). The one or more image sensors 1 may provide a relay of information regarding an action area where a physician manipulates tissue with at least one surgical instrument 300 during a surgical procedure, with the action area located outside of the bore 116 and radially between the edges 121 of the sidewall 119. In a particular embodiment, the area between the edges 121 is entirely above the opening into the bore 116. As seen in FIG. 1A, the sidewall 119 extends radially to form a semi-rectangular shape with an open top area between the axially-extending edges 121 of the sidewall 119. It should be understood, however, that the sidewall 119 could be formed in other shapes, including a closed circle, semi-circle, hexagonal, etc., and may include openings at radial positions other than or in addition to the top opening. In general, the tubular body portion 126 forms a surface for supporting and guiding a surgical implement 300, which may be a cutting instrument, in some manner.

Referring again to FIG. 1A, embodiments of the surgical device 1000 may include a cannula 100 having a bore 116 as described above, in which the bore 116 may feature an orthogonal or cylindrical shape as defined by the sidewall 119 that extends axially and radially and that includes a pair of axially-extending edges 121.

In an exemplary embodiment, the support surface 110 at the distal end 114 of the cannula 100 is an extension of the sidewall 119 such that a closed tubular portion is formed. In some embodiments, the support surface 110 is a flat surface extending in a direction perpendicular to the axial direction. In the illustrated embodiment, the support surface 110 also extends along the axially-extending edges 121 of the sidewall 119 in an axial direction. The support surface 110 also provides a stop for the surgical implement 300. In another embodiment, the support surface 110 may include an aperture 111 to enable the image sensor(s) 1 to have an axial line of sight through the aperture 111. The diameter of the aperture 111 may be as large or as small as needed to accommodate an axial line of sight for the image sensor(s) 1, through the aperture 111.

Further, the cannula 100 of the surgical device 1000 may include the base member 200 at the proximal end 112. As will be described in more detail, the base member 200 may define a generic opening into the bore 116 within the tubular body 126 portion for slidably receiving at least one surgical implement 300, such as a forward cutting knife, reverse cutting knife or rasp, for example, and the assembly 10 including the one or more image sensors (optical sensors) 1. As used herein, the term generic means that the opening is not side specific and it can accommodate tools for surgical procedures configured for a patient's left or right side. The base member 200 further includes a pair of wings 125 which extend in a direction perpendicular to the axial direction and provide a flat surface at a lower portion of the base member 200. This flat surface rests on the patient's body, helping to stabilize the cannula 100 during use. In addition, the upper portion 115 of the base member 200 may be a planar surface, as shown in FIG. 1A. In another embodiment, and in contrast to the upper portion 115 of the base member 200 shown in FIG. 1A, the base member 200 may instead feature a configuration with hubs that allow a physician to grip the cannula 100 more easily during use.

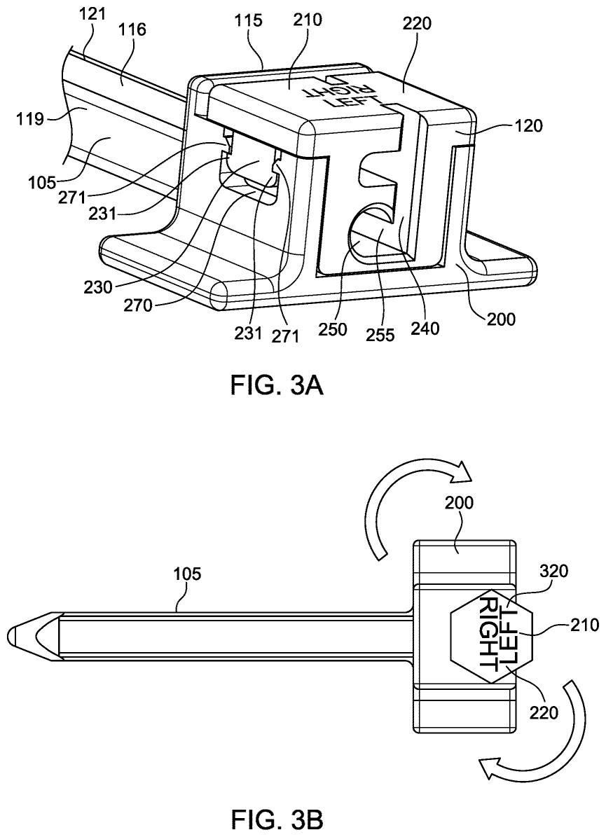

In addition, the surgical device 1000 may also accommodate an obturator 118 (as shown in FIG. 3C) that may be positioned in the right or left orientation and may be slidably inserted through the generic opening of the base member 200 and into the bore 116 within the tubular body portion 126 of the cannula 100. The obturator 118 provides for rigidity during the initial insertion of the device 1000 into the surgical incision and also prevents other soft tissue anatomy from entering into the bore 116 between the axially-extending edges 121 of the sidewall 119. In addition to the obturator 118, the surgical device 1000 may also accommodate a configurable guide insert 120 (as shown in FIG. 3A) that may be positioned in one of two orientations. As will be subsequently described in more detail, the configurable guide insert 120 may be inserted into and rigidly attached to the cannula 100 at an engagement point adjacent to the generic opening of the base member 200.

FIG. 1A thus show an exploded view of the surgical device 1000 as it may be used for the initial insertion at the surgical incision. In the initial insertion of the device 1000, the distal end 114 of the cannula 100 is inserted first in an incision. The support surface 110 contacts soft tissue structures at the surgical sight, providing support and inhibiting the soft tissue structures from blocking the bore 116, thereby limiting the potential of the soft tissue structures from contacting or impeding the view or motion of the surgical implements. Likewise, the obturator 118 (as shown in FIG. 3C) prevents the entry of the soft tissue structures within the bore 116 of the tubular body 126. The cannula 100 may be formed from suitable material which is acceptable for being temporarily inserted into the human body during a surgical procedure. For example, the cannula 100 may be formed from a medically-acceptable plastic material. Metal materials are also possible. The cannula 100 may be manufactured using an opaque, translucent, or transparent material.

Referring again to FIG. 1A in conjunction with FIG. 1B, embodiments of the surgical device 1000 may include an assembly 10 including the image sensors (optical sensors) 1, housing 4, and additional optical imaging components 2, 3, 5, 6, 7 that are permanently fixed within the cannula 100, as shown in FIG. 1B. In such embodiments, the assembly 10 with housing 4 may reside within the tubular body portion 126 that extends axially from the base member 200 to the support surface 110 of the end portion 109. As shown in FIG. 1B, the housing 4 of the assembly 10 may reside in a fixed position within the bore 116 of the cannula 100 tangentially between the pair of axially-extending edges 121 of the sidewall 119. As further shown in FIG. 1B, the housing 4 of the assembly 10 that is fixed within the bore 116 of the cannula 100 may also encase additional optical imaging components, including a light source 2 such as an LED, and a rigid or semi-rigid conduit 5 for enclosing the flexible circuitry 3.

Referring again to FIG. 1A in conjunction with FIG. 1B, the assembly 10 that is fixed within the cannula 100 may be hermetically sealed by the housing 4 to prevent introduction of contaminants into the assembly 10. For example, the semi-rigid conduit 5 for enclosing the flexible circuitry 3 and the housing 4 may be contiguous with each other and with the coupler 6, so that these components 4, 5, 6 hermetically seal the optical imaging components 1, 2, 3, 7 of the assembly 10 so that contaminants cannot compromise the assembly 10 and so that the assembly 10 and optical imaging components may be reusable. Although the housing 4 as shown in FIG. 1A encases the image sensors 1 and LEDs 2, in some embodiments the housing 4 may be configured to encase additional optical imaging components including the flexible circuitry 3, and the semi-rigid conduit 5. In an embodiment, the assembly 10 may be disposable. In another embodiment, one or all of the housing 4, the semi-rigid conduit 5, and the coupler 6 may be disposable. In other embodiments, the one or more image sensors 1, the one ore more LEDs 2, the flexible circuitry 3, and/or the connection mechanism 7 for optical image transfer to another device may be disposable. An assembly 10 including the image sensors (optical sensors) 1, housing 4, and additional optical imaging components 2, 3, 5, 6, 7 may be disposable with or without the cannula 100 being disposable. In addition, it is to be understood that one or more of the optical imaging components as described herein may be manufactured and assembled according to a 3D printing or additive manufacturing process.

Referring again to FIG. 1A in conjunction with FIG. 1B, embodiments of the surgical device 1000 may include a cannula 100 having a bore 116 as described herein, in which the bore 116 may feature an orthogonal or cylindrical shape as defined by the contours of the upper surface 45 of the housing 4 and the contours of the sidewall 119 that extends axially and radially and that includes a pair of axially-extending edges 121. Although the embodiments of FIG. 1B shows a bore 116 featuring an orthogonal shape, it is to be understood that the bore 116 may feature a cylindrical shape as defined by the contours of the upper surface 45 of the housing 4 and the contours of the sidewall 119. In embodiments in which the bore 116 may feature a cylindrical shape as defined by the contours of the upper surface 45 of the housing 4 and the contours of the sidewall 119, an upper surface 45 of the housing 4 may feature a concave shape.

Referring again to FIG. 1B, it is to be understood that the surgical device 1000 may include an ability to wirelessly transmit image data from the image sensors 1 to a display, so that the connection mechanism for optical image transfer to another device may not be necessary. For example, when the assembly 10 and image sensors 1 include wireless functionality, it may not be necessary to include all of the additional optical imaging components within the housing 4, such as the flexible circuitry 3 for carrying the captured image(s) to a coupler 6, a rigid or semi-rigid conduit 5 for enclosing the flexible circuitry 3, the coupler 6 for connection to an optical display (not shown in FIG. 1B), and the connection mechanism 7 for optical image transfer to another device.

Referring again to FIG. 1B, in embodiments of the surgical device 1000 in which the assembly 10 is permanently fixed within the cannula 100, the one or more image sensors 1 may be embedded within the housing 4 of the assembly, so that the sensors 1 may be flush with an upper surface 45 of the housing 4, as shown in FIG. 1B. The sensors 1 may feature a small form factor that may be square or rounded shape. A sensor 1 may have a small form factor of approximately 1 millimeter in length, or 1 square millimeter, so that it may conveniently embed within the assembly 10 housing 4 and accompany the size of the cannula 100 including the dimensions of the bore 116 and sidewall 119. With the assembly 10 permanently fixed within the cannula, the upper surface 45 of the housing 4 (as shown in FIG. 1B) may feature a plane extending axially along a length the tubular body portion 126, so that the upper surface 45 may reside within the bore 116 of the cannula 100 tangentially between the pair of axially-extending edges 121 of the sidewall 119. The embedded sensors 1 within the assembly 10 housing 4 may be flush with the plane of the upper surface 45 of the housing 4 and encased within the housing member 4. The embedded image sensors 1 may be arranged in a defined array of image sensors 1, such as in a cluster or pattern about the upper surface 45 of the housing 4, as shown in FIG. 1B. A cannula 100 including multiple image sensors 1 may display either a continuous single image or combination of images from various image sensors 1 and vantages points. In addition, the image sensors 1 as shown in FIG. 1B may be adjacent to one or more light sources 2 such as LED light sources. One or more light sources 2 such as LEDs may reside within the assembly 10 housing 4 and be flush with or protrude through the upper surface 45 of the housing 4.

Referring again to FIG. 1B, in other embodiments, the embedded image sensors 1 may be sub-flush with the plane of the upper surface 45 of the housing 4 and encased within the housing member 4. In particular, when the embedded image sensors 1 are sub-flush with the plane of the upper surface 45 of the housing 4, there might be a plastic layer or plastic portion between the embedded image sensors 1 and the plane of the upper surface 45 of the housing 4. When there is a plastic layer or plastic portion between the embedded image sensors 1 and the plane of the upper surface 45 of the housing 4, the image sensors 1 may be below the upper surface 45 of the housing, so that the image sensors 1 are embedded within a height of the housing member 4 and not flush with the upper surface 45 of the housing. The plane of the upper surface 45 of the housing 4 may be exposed to the surgical instruments that may slidably move across the plane of the upper surface 45 of the housing 4. For example, the one or more of the embedded image sensors 1 may be sub-flush with the plane of the upper surface 45 of the housing 4 so that the embedded image sensors 1 may be embedded within the housing 4 and do not protrude outwardly from the plane of the upper surface 45 of the housing 4.

Referring again to FIG. 1B, in other embodiments, the embedded image sensors 1 may protrude through the plane of the upper surface 45 of the housing 4. The plane of the upper surface 45 of the housing 4 may be exposed to the surgical instruments that may slidably move across the plane of the upper surface 45 of the housing 4. For example, the one or more of the embedded image sensors 1 may protrude through the plane of the upper surface 45 of the housing 4 so that the embedded image sensors 1 may not necessarily be entirely encased within the housing 4. When the embedded image sensors 1 may protrude outwardly through the plane of the upper surface 45 of the housing 4, the surgeon may slidably move the surgical instruments across the plane of the upper surface 45 of the housing 4 and adjacent to the protruding image sensors 1, so that the image sensors 1 may protrude in a position adjacent to the smooth movement of the surgical instrument along the plane of the upper surface 45 of the housing 4. When the embedded image sensors 1 protrude through the plane of the upper surface 45 of the housing 4, the embedded image sensors 1 may be exposed to the bore 116 of the cannula 100.

Referring again to FIG. 1B, in embodiments of the surgical device 1000 in which the assembly 10 is permanently fixed within the cannula 100, because the sensors 1 may be embedded within the assembly 10 housing 4, the sensors 1 may be flush with the upper surface 45 of the housing 4. As shown in FIG. 1B, the sensors 1 may not protrude outwardly from the upper surface 45 of the housing 4. When such sensors 1 are embedded and flush with the upper surface 45 of the assembly 10 housing 4, the surgeon may slidably move a surgical instrument 300, such as a cutting tool or scalpel, across the upper surface 45 of the housing 4 that includes the embedded sensors 1 without bumping the embedded sensors 1. Inner planes of the cannula 100, which may be touched or in contact with a surgical instrument 300, may include the sidewall 119 of the axially-extending edges 121 of the cannula 100.

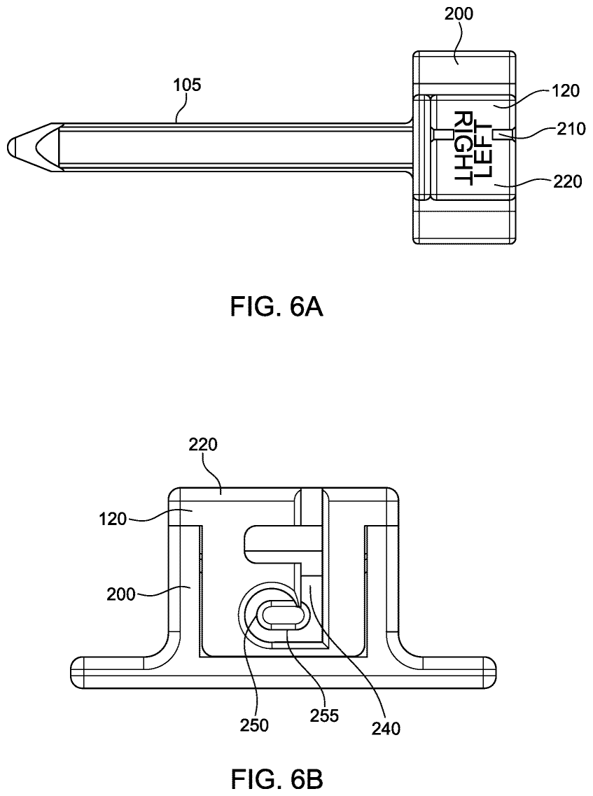

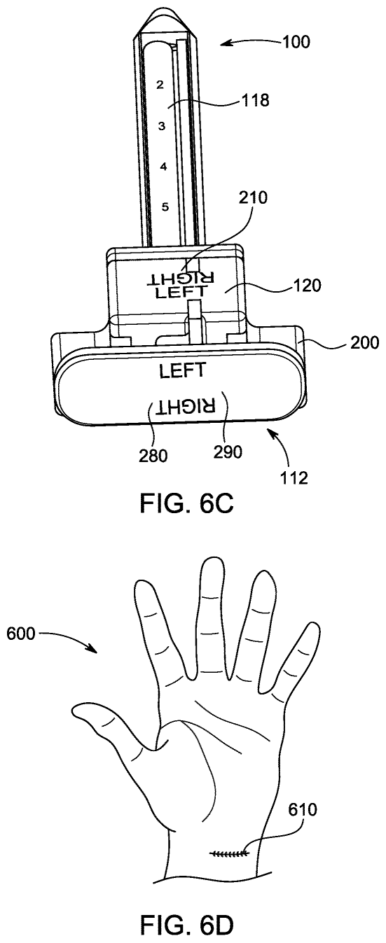

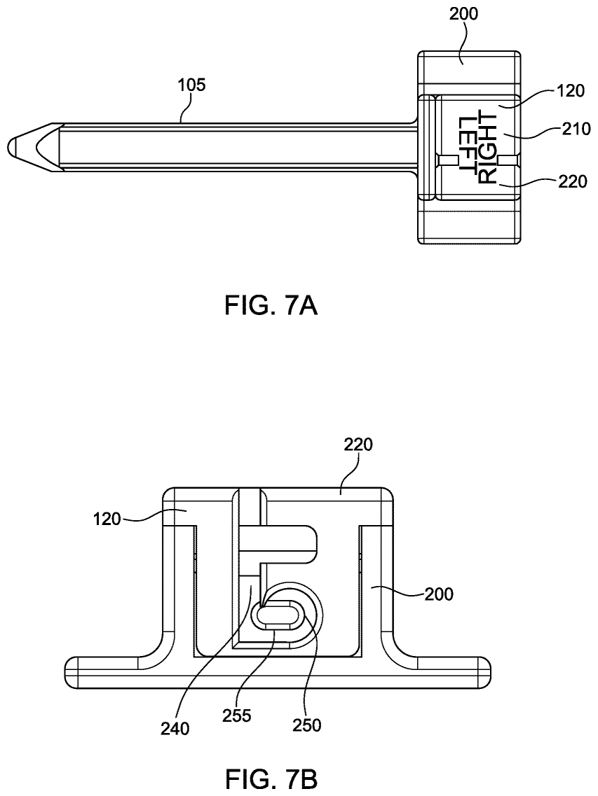

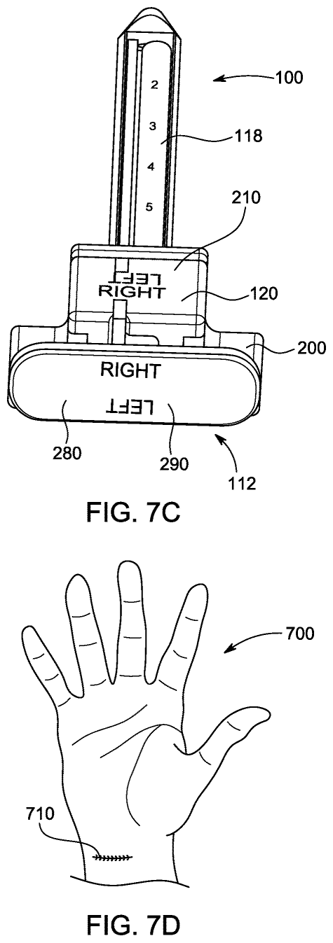

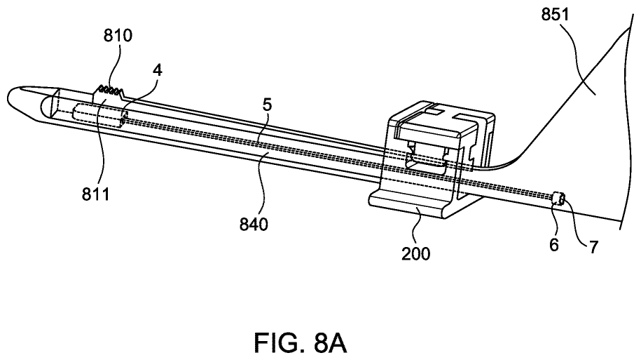

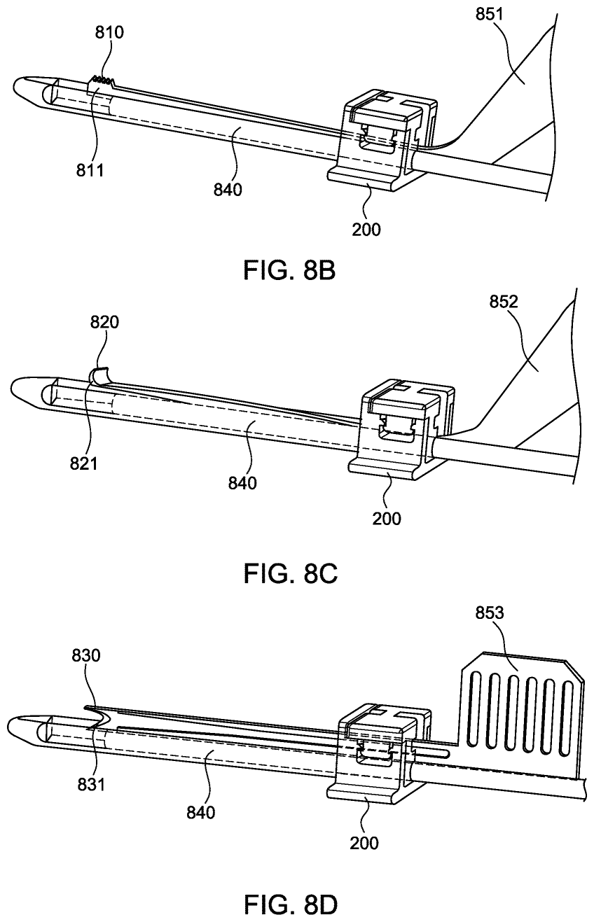

Referring again to FIG. 1A in conjunction with FIG. 8A, embodiments of the surgical device 1000 may also include an assembly 10 including one or more image sensors (optical sensors) 1 and the additional optical imaging components 2, 3, 4, 5, 6, 7, 8, that is permanently fixed to a lower portion of the cannula 100 while also having additional capability for slidable movement and adjustable positioning within the cannula 100 along a longitudinal axis of the cannula 100. For example, the cannula 100 may feature fixed position engagement points 800 to secure the assembly 10 at defined intervals along the cannula 100. In such embodiments, the assembly 10 housing 4 may also feature one or more fixed position engagement portions 8. The fixed position engagement portions 8 may each be a protrusion from the housing 4 of the assembly 10 that fits and matingly couples within any of the fixed position engagement points 800 of the cannula 100. As understood by those of skill in the art, a fixed position engagement portion 8 may be spring loaded and manually controlled by the physician from a proximal end 112 of the device 1000 in order to incrementally advance the assembly 10 distally or proximally in an axial direction within the cannula 100, as shown in FIG. 8A. For example, and referring again to FIG. 8A in conjunction with FIG. 1A, when a physician advances the assembly 10 housing 4 featuring one or more fixed position engagement portions 8, the fixed position engagement portions 8 may contact and pass along an interior of a sidewall 119 having the fixed position engagement points 800 of the cannula 100. When the one or more fixed position engagement portions 8 are in contact with the sidewall 119, they do not protrude from the housing 4 until they align with the fixed position engagement points 800, so that the fixed position engagement portions 8 may then spring or pop into the fixed position engagement points 800 of the cannula 100 to secure an incremental position of the assembly 10 housing 4 within the cannula 100. Together, the fixed position engagement portions 8 and the fixed position engagement points 800 may enable the physician to adjust and secure a selectable incremental positioning of one or more sensors 1 within the cannula 100. As physician advances the assembly 10 distally or proximally in an axial direction within the cannula 100, the physician may select a desired incremental positioning of the sensors 1 within the cannula 100 to obtain an optimum visualization of a target tissue, for example. When the one or more sensors 1 is movable within the cannula 100, the one or more sensors 1 may move axially about the cannula 100. In other embodiments, when the one or more sensors 1 is movable within the cannula, the one or more sensors 1 may be configured to move axially and/or circumferentially about the cannula 100. The device 1000 provides the physician with an ability to place the device 1000 at an incision point 610, 710 (as shown in FIGS. 6D and 7D) and advance or retract one or both of the assembly 10 (with one or more image sensors 1) and a surgical instrument 300 along an axis of the cannula 100 when both the image sensors 1 and the surgical instrument 300 are present at the incision point 610, 710.

In such embodiments of the surgical device 1000 in which the assembly 10 may be permanently fixed to a lower portion of the cannula 100 while also having additional capability for slidable movement and adjustable positioning within the cannula 100 along a longitudinal axis of the cannula 100, the assembly 10, while fixed within the cannula 100, may slidably reside within the tubular body portion 126 that extends axially from the base member 200 to the support surface 110 of the end portion 109. The assembly 10 that is fixed within the cannula 100 may be hermetically sealed by the housing 4. The housing 4 of the assembly 10 may reside within the bore 116 of the cannula 100 tangentially between the pair of axially-extending edges 121 of the sidewall 119. The physician may slidably advance the assembly 10 (as shown in FIG. 8A) in a distal direction along the cannula 100 and slidably advance the assembly 10 (as shown in FIG. 8A) in a proximal direction along the cannula 100. The assembly 10 may also include the additional optical imaging components including a flexible circuitry 3 for carrying the captured image(s) from the image sensor to a coupler 6, a rigid or semi-rigid conduit 5 for enclosing the flexible circuitry 3, a coupler 6 for connection to an optical display (not shown in FIG. 1A), and a connection mechanism 7 for optical image transfer to another device.

When advancing the assembly 10 (as shown in FIG. 1A in conjunction with FIG. 8A) in a distal direction along the cannula 100, the physician may advance a portion of the additional optical imaging components that are connected to the housing 4, such as the flexible circuitry 3 and the rigid or semi-rigid conduit 5 for enclosing the flexible circuitry 3 through a slot 400 in the base member 200. Based on the combination of an engagement portion 211 that provide a mechanism for acceptance of a surgical instrument 300, and the slot 400 that accommodates a slidable movement of the flexible circuitry 3 and the rigid or semi-rigid conduit 5 through the slot 400 in the base member 200, the surgical device 1000 allows for motion of the surgical instrument 300 and the one or more image sensors 1 when both are present.

In an embodiment of the surgical device 1000 in which the assembly 10 may be permanently fixed to a lower portion of the cannula 100 while also having additional capability for slidable movement and adjustable positioning within the cannula 100 along a longitudinal axis of the cannula 100, the surgical instrument 300 as shown in FIG. 1A may also engage with the assembly 10 to advance the assembly 10 along an axial length of the cannula 100 or retract the assembly 10 along an axial length of the cannula 100. In another embodiment, the assembly 10 may attach to the surgical instrument 300 (or another surgical instrument) providing a fixed relationship between the assembly 10 and the surgical instrument 300. In such an embodiment, the assembly 10 may not need to be confined within the slot 400 in the base member 200 because the surgical instrument 300 may utilize an engagement mechanism between the groove 310 on the surgical instrument 300 and the engagement portion 211 for acceptance of the surgical instrument 300.

As shown in FIG. 1A, the slot 400 may be below an engagement portion 211 for acceptance of a surgical instrument 300, such as a cutting tool or scalpel. Although FIG. 1A shows the slot 400 below the engagement portion 211 in a top-to-bottom relationship, it is understood that the slot 400 may be on a left or right side of the engagement portion 211 in a side-to-side relationship in the base member 200. With the fixed position engagement portions 8 and the fixed position engagement points 800, the physician may adjust the positioning of one or more sensors 1 within the cannula 100 by manipulating the assembly 10 with pulling or pushing through the slot 400. In such an embodiment in which the assembly 10 is permanently fixed to the cannula 100 while also having additional capability for slidable movement and adjustable positioning within the cannula 100, the assembly 10 may not be entirely removable from the cannula 100 through the slot 400. For example, while the slot 400 may be large enough to accommodate slidable movement of the flexible circuitry 3 and the rigid or semi-rigid conduit 5 through the slot 400 in the base member 200, the slot 400 may be configured with dimensions that do not accommodate slideable movement of the housing 4 of the assembly 10 through the slot 400. As such, the housing 4 portion of the assembly 10 may slide axially within the bore 116 of the tubular body 126 between the end portion 109 and the upper portion 115 of the base member 200, so that a physician may position the one or more sensors 1 incrementally by utilizing the fixed position engagement portions 8 and the fixed position engagement points 800. The physician may adjust a positioning of one or more sensors 1 within the cannula 100, so that the physician may obtain an optimum visualization of a target tissue.

In embodiments of the surgical device 1000 in which the assembly 10 may be permanently fixed to a lower portion of the cannula 100 while also having additional capability for slidable movement and adjustable positioning within the cannula 100 along a longitudinal axis of the cannula 100, the one or more image sensors 1 may be embedded within the housing 4 of the assembly 10, so that the sensors 1 may be flush with an upper surface 45 of the housing 4, as shown in FIG. 1B, for example. The sensors 1 may feature a small form factor that may be square or rounded shape. A sensor 1 may have a small form factor of approximately 1 millimeter in length, or 1 square millimeter, so that it may conveniently embed within the assembly 10 housing 4 and accompany the size of the cannula 100 including the dimensions of the bore 116 and sidewall 199. With the assembly 10 slidably fixed within the cannula 100, the upper surface 45 of the housing 4 may feature a plane extending axially along a length the tubular body portion 126, so that the upper surface 45 may reside within the bore 116 of the cannula 100 tangentially between the pair of axially-extending edges 121 of the sidewall 119. The embedded sensors 1 within the assembly 10 housing 4 may be flush with the plane of the upper surface 45 of the housing 4 and encased within the housing member 4. In addition, the image sensors 1 as shown in FIG. 1B may be adjacent to one or more light sources 2 such as LED light sources. One or more light sources 2 such as LEDs may reside within the assembly 10 housing 4 and be flush with or protrude through the upper surface 45 of the housing 4.

In other embodiments of the surgical device 1000 in which the assembly may be permanently fixed to a lower portion of the cannula 100 while also having additional capability for slidable movement and adjustable positioning within the cannula 100 along a longitudinal axis of the cannula 100, the one or more image sensors 1 may be embedded within the housing 4 of the assembly 10, so that the sensors 1 may be sub-flush with an upper surface 45 of the housing 4. For example, the embedded image sensors 1 may be sub-flush with the plane of the upper surface 45 of the housing 4 and encased within the housing member 4 of the assembly 10. In particular, when the embedded image sensors 1 are sub-flush with the plane of the upper surface 45 of the housing 4, there might be a plastic layer or plastic portion between the embedded image sensors 1 and the plane of the upper surface 45 of the housing 4. When there is a plastic layer or plastic portion between the embedded image sensors 1 and the plane of the upper surface 45 of the housing 4, the image sensors 1 may be below the upper surface 45 of the housing, so that the image sensors 1 are embedded within a height of the housing member 4 and not flush with the upper surface 45 of the housing. The plane of the upper surface 45 of the housing 4 may be exposed to the surgical instruments that may slidably move across the plane of the upper surface 45 of the housing 4. For example, the one or more of the embedded image sensors 1 may be sub-flush with the plane of the upper surface 45 of the housing 4 so that the embedded image sensors 1 may be embedded within the housing 4 and do not protrude outwardly from the plane of the upper surface 45 of the housing 4.

In other embodiments of the surgical device 1000 in which the assembly may be permanently fixed to a lower portion of the cannula 100 while also having additional capability for slidable movement and adjustable positioning within the cannula 100 along a longitudinal axis of the cannula 100, the one or more image sensors 1 may be embedded within the housing 4 of the assembly 10, so that the sensors 1 may protrude through the plane of the upper surface 45 of the housing 4. The plane of the upper surface 45 of the housing 4 may be exposed to the surgical instruments that may slidably move across the plane of the upper surface 45 of the housing 4. For example, the one or more of the embedded image sensors 1 may protrude through the plane of the upper surface 45 of the housing 4 so that the embedded image sensors 1 may not necessarily be entirely encased within the housing 4. When the embedded image sensors 1 may protrude outwardly through the plane of the upper surface 45 of the housing 4, the surgeon may slidably move the surgical instruments across the plane of the upper surface 45 of the housing 4 and adjacent to the protruding image sensors 1, so that the image sensors 1 may protrude in a position adjacent to the smooth movement of the surgical instrument along the plane of the upper surface 45 of the housing 4. When the embedded image sensors 1 protrude through the plane of the upper surface 45 of the housing 4, the embedded image sensors 1 may be exposed to the bore 116 of the cannula 100.

In embodiments of the surgical device 1000 in which the assembly 10 is permanently fixed within the cannula 100, because the sensors 1 may be embedded within the assembly 10 housing 4, the sensors 1 may be flush with the upper surface 45 of the housing 4, so that the sensors 1 do not protrude outwardly from the upper surface 45 of the housing 4. When such sensors 1 are embedded and flush with the upper surface 45 of the assembly 10 housing 4, the surgeon may slidably move a surgical instrument 300, such as a cutting tool or scalpel, across the upper surface 45 of the housing 4 that includes the embedded sensors 1 without bumping the embedded sensors 1. Inner planes of the cannula 100, which may be touched or in contact with a surgical instrument 300, include the sidewall 119 of the axially-extending edges 121 of the cannula 100.

Referring again to FIG. 1A, embodiments of the surgical device 1000 may also include an assembly 10, including the one or more image sensors (optical sensors) 1 and the additional optical imaging components 2, 3, 4, 5, 6, 7, 8, that is entirely removably from the cannula 100, Such an embodiment of the surgical device 1000 may be similar to the previously described embodiment of the surgical device 1000 in which the assembly 10 may be permanently fixed to a lower portion of the cannula 100 while also having additional capability for slidable movement and adjustable positioning within the cannula 100 along a longitudinal axis of the cannula 100. However, in an embodiment of the surgical device in which an assembly 10 including the one or more image sensors (optical sensors) 1 is entirely removably from the cannula 100, the slot 400 in the base member 200 may be configured with dimensions that also accommodate slideable movement of the housing 4 of the assembly 10 through the slot 400, so that the assembly 10 including the housing 4 may slide along a length of the cannula 100 and be entirely removed from the cannula 100 through the slot 400 of the base member 200.

Referring again to FIG. 1A, in embodiments of the surgical device 1000 that include an assembly 10 including the one or more image sensors (optical sensors) 1 that is entirely removably from the cannula 100, the cannula 100 may feature the fixed position engagement points 800 to secure the assembly 10 at defined intervals along the cannula 100. In such embodiments, the assembly 10 housing 4 may also feature one or more fixed position engagement portions 8. The fixed position engagement portions 8 may each be a protrusion from the housing 4 of the assembly 10 that fits within any of the fixed position engagement points 800 of the cannula 100. As understood by those of skill in the art, a fixed position engagement portion 8 may be spring loaded and manually controlled by the physician from a proximal end 112 of the device 1000 in order to incrementally advance the assembly 10 distally or proximally within the cannula 100, as shown in FIG. 8A. For example, and referring again to FIG. 8A in conjunction with FIG. 1A, when a physician advances the assembly 10 housing 4 featuring one or more fixed position engagement portions 8, the fixed position engagement portions 8 may contact and pass along an interior of a sidewall 119 having the fixed position engagement points 800 of the cannula 100. When the one or more fixed position engagement portions 8 are in contact with the sidewall 119, they do not protrude from the housing 4 until they align with the fixed position engagement points 800, so that when aligned, the fixed position engagement portions 8 may then spring or pop into the fixed position engagement points 800 of the cannula 100 to secure an incremental position of the assembly 10 housing 4 within the cannula 100. Together, the fixed position engagement portions 8 and the fixed position engagement points 800 may enable the physician to adjust a positioning of one or more sensors 1 within the cannula 100, so that the physician may obtain an optimum visualization of a target tissue, for example.

Referring again to FIG. 1A, in embodiments of the surgical device 1000 that include an assembly 10, including one or more image sensors (optical sensors) 1, that is entirely removably from the cannula 100, when advancing the assembly 10 (as shown in FIG. 1A in conjunction with FIG. 8A) in a distal direction along the cannula 100, the physician may insert and advance the assembly 10 with housing 4, along with the additional optical imaging components that are connected to the housing 4, such as the flexible circuitry 3 and the rigid or semi-rigid conduit 5 for enclosing the flexible circuitry 3, through the slot 400 in the base member 200. Based on the combination of an engagement portion 211 for acceptance of a surgical instrument 300 and the slot 400 that accommodates a slidable movement of the assembly 10, including the housing 4, the one or more sensors 1, the LED lights 2, the flexible circuitry 3, and the rigid or semi-rigid conduit 5, through the slot 400 in the base member 200, the surgical device 1000 allows for motion of the surgical instrument 300 and the one or more image sensors 1 when both are present.

In an embodiment of the surgical device 1000 in which an assembly 10 including the one or more image sensors (optical sensors) 1 is entirely removably from the cannula 100, the surgical instrument 300 as shown in FIG. 1A may also engage with the assembly 10 to advance the assembly 10 along an axial length of the cannula 100 or retract the assembly 10 along an axial length of the cannula 100. In another embodiment, the assembly 10 may attach to the surgical instrument 300 (or another surgical instrument) providing a fixed relationship between the assembly 10 and the surgical instrument 300. In such an embodiment, the assembly 10 may not need to be confined within the slot 400 in the base member 200 because the surgical instrument 300 may utilize an engagement mechanism between the groove 310 on the surgical instrument 300 and the engagement portion 211 for acceptance of the surgical instrument 300.

Referring again to FIG. 1A, in embodiments of the surgical device 1000 with an assembly 10, including the one or more image sensors (optical sensors), 1 that is entirely removably from the cannula 100, the slot 400 may be below an engagement portion 211 for acceptance of a surgical instrument 300, such as a cutting tool or scalpel. Although FIG. 1A shows the slot 400 below the engagement portion 211 in a top-to-bottom relationship, it is understood that the slot 400 may be on a left or right side of the engagement portion 211, in a side-to-side relationship in the base member 200. With the one or more fixed position engagement portions 8 and the fixed position engagement points 800, the physician may adjust the positioning of one or more sensors 1 within the cannula 100 by manipulating the assembly 10 with pulling or pushing through the slot 400. In such an embodiment, the assembly 10 is entirely removable from the cannula 100 while also having additional capability for slidable movement and adjustable positioning within the cannula 100. The slot 400 may be large enough to accommodate slidable movement of the assembly 10 and housing 4, in addition to the flexible circuitry 3 and the rigid or semi-rigid conduit 5, through the slot 400 in the base member 200.

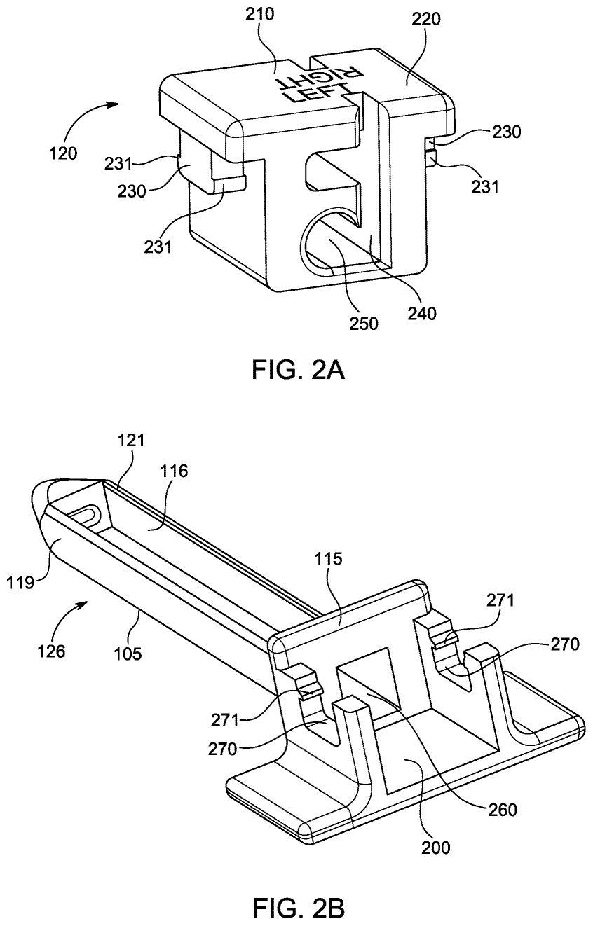

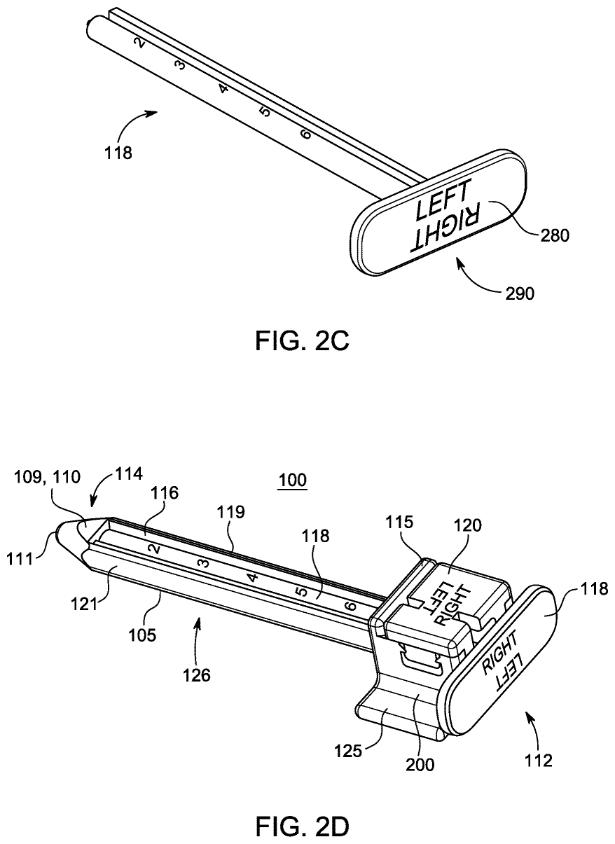

As will be described in further detail in reference to the subsequent Figures and related description, additional embodiments of the surgical device 1000 with an assembly 10 including the one or more image sensors (optical sensors) 1 that is entirely removably from the cannula 100 may include a configuration utilizing a configurable guide insert 120, as shown in FIG. 2A, for example.

Referring to FIGS. 2A-2D, a configurable guide insert 120 is shown that may be utilized with embodiments of the surgical device 1000 with an assembly 10 including the one or more image sensors (optical sensors) 1 that is entirely removably from the cannula 100. In FIGS. 2A-2C. In FIGS. 2A-2C, three components of the device 1000 are depicted individually. As will be described in further detail, FIG. 2A shows a configurable guide insert 120, FIG. 2B shows the cannula 100 with the base member 200, FIG. 2C shows an obturator 118 of the device 1000, and FIG. 2D shows a side-perspective view of the surgical device 1000 in which the configurable guide insert 120 and obturator 118 are in place within the cannula 100.

The configurable guide insert 120, as shown in FIG. 2A, may feature a singular contiguous opening including an opening 250 and a slot opening 240. The configurable guide insert 120 may further include a top portion 220 with markings 210, and engaging members 230 for rigidly attaching to the cannula 100 at corresponding receiving points 270 (as shown in FIG. 2B) in the base member 200. In one embodiment, the engagement members 230 on both sides of the configurable guide insert 120 are identical to each other. The receiving points 270 may include ridges 271 that couple to the corresponding ridges 231 of the engaging members 230 of the configurable guide insert 120.

The corresponding ridges 231 may be formed as an enlarged head portion of the engaging members 230, such that the engagement members have a T-shaped profile. The enlarged head portion of the engaging members 230 may be press-fit, snap-fit, or interference fit into engagement with the ridges 271 of the receiving points 270. The connection between the configurable guide insert 120 and the base member 200 of the cannula 100 is achieved via direct connection between the receiving points 270 and corresponding ridges 231. The term receiving points is used generally herein to generally refer to any shaped receptacle or opening in the base member 200. The term corresponding ridges 231 is used generally herein to refer to any shaped protrusion or arm formed on the configurable guide insert 120. Although specific geometries and configurations are illustrated in the drawings, one of ordinary skill in the art would understand based on this disclosure that any complementary shaped interface can be provided between the configurable guide insert 120 and the base member 200. No additional fastening or securing elements are required to attach the configurable guide insert 120 to the cannula 100 besides the integrally formed features formed on each of these components.

As aforementioned, the configurable guide insert 120, as shown in FIG. 2A, may feature a singular contiguous opening including an opening 250 and a slot opening 240, which define axially-extending spaces for receiving other components of the surgical device 1000, thereby accommodating insertion of multiple surgical instruments. The opening 250 and slot opening 240 define the designated regions within the singular contiguous opening of the configurable guide insert 120 for the restricted positioning and movement of the surgical instruments within the configurable guide insert 120.

In an embodiment, the opening 250 may receive an assembly 10 including the one or more image sensors (optical sensors) 1 (as shown in FIG. 1A) that is entirely removably from the cannula 100. The slot opening 240 may receive a surgical implement 300, such as a cutting tool or scalpel. The assembly 10 and surgical instrument 300 are restricted to only stay within their respective designated regions within the singular contiguous opening through the configurable guide insert 120. More specifically, the opening 250 provides an opening through the configurable guide insert 120 for insertion of an assembly 10 including the image sensor 1, housing 4, and additional optical imaging components that are connected to the housing 4, such as the flexible circuitry 3 and the rigid or semi-rigid conduit 5 for enclosing the flexible circuitry 3, and the slot opening 240 provides an opening through the configurable guide insert 120 for insertion of a surgical instrument 300, such as a cutting implement or rasp, for example.

While specific geometries and configurations are illustrated in the drawings for the opening 250 and a slot opening 240, one of ordinary skill in the art would understand based on this disclosure that any complementary shaped interface may be respectively provided between the opening 250 and a slot opening 240 of the configurable guide insert 120 and the assembly 10 and surgical instrument 300. For example, it is understood that the opening 250, as shown in FIG. 2A, may feature a different shape, such as the rectangular shape of the slot 400 for receiving the image sensor 1 and housing 4, as shown in FIG. 1A.

As aforementioned, the opening 250 of the configurable guide insert 120 may receive an assembly 10 with housing 4 including the one or more image sensors (optical sensors) 1 (as shown in FIG. 1A) that is entirely removably from the cannula 100. Accordingly, in an embodiment, the shape of the assembly 10 housing 4 may be orthogonal (as shown in FIG. 1A), and the shape of the bore 116 may accommodate the orthogonal assembly 10 housing 4 with a corresponding orthogonal shape, as defined by the contours of the upper surface 45 of the housing 4 and the contours of the sidewall 119. In such an embodiment, when a physician advances or retracts the assembly 10 housing 4 through the opening 250 of the configurable guide insert 120 into the bore 116 of the cannula 100, the shape of the assembly 10 housing 4 corresponds and fits within the orthogonally shaped bore 116. It is understood that the dimensions of the bore 116 as defined by the contours of the upper surface 45 of the housing 4 and the contours of the sidewall 119 may accommodate the dimensions of the assembly 10 housing 4, so that the physician may advance or retract the assembly 10 housing 4 through the opening 250 of the configurable guide insert 120 into the bore 116 of the cannula 100. In another embodiment, the shape of the assembly 10 housing 4 may be cylindrical, and the shape of the bore 116 may accommodate the cylindrical assembly 10 housing 4 with a corresponding cylindrical shape, as defined by the contours of the upper surface 45 of the housing 4 and the contours of the sidewall 119. In such an embodiment, when a physician advances or retracts the assembly 10 housing 4 through the opening 250 of the configurable guide insert 120 into the bore 116 of the cannula 100, the shape of the assembly 10 housing 4 corresponds and fits within the cylindrically shaped bore 116. It is understood that the dimensions of the bore 116 as defined by the contours of the upper surface 45 of the housing 4 and the contours of the sidewall 119 may accommodate the dimensions of the assembly 10 housing 4, so that the physician may advance or retract the assembly 10 housing 4 through the opening 250 of the configurable guide insert 120 into the bore 116 of the cannula 100.

Although the shape of the opening 250 of the configurable guide insert 120 as shown in FIGS. 2A, 3A, 4A, 5A, 6B, and 7B is round, it is further understood that the shape of the opening 250 of the configurable guide insert 120 may also accommodate embodiments of the device 1000 in which the shape of the assembly 10 housing 4 may be orthogonal (as shown in FIG. 1A) with the shape of the bore 116 also featuring a corresponding orthogonal shape. In such embodiments, the shape of the opening 250 of the configurable guide insert 120 may also be orthogonal to accommodate and receive an assembly 10 with housing 4 including the one or more image sensors (optical sensors) 1 (as shown in FIG. 1A) that is entirely removably from the cannula 100. It is also understood that the shape of the opening 250 of the configurable guide insert 120 may also accommodate embodiments of the device 1000 in which the shape of the assembly 10 housing 4 may be cylindrical with the shape of the bore 116 also featuring a corresponding cylindrical shape. In such embodiments, the shape of the opening 250 of the configurable guide insert 120 may also be cylindrical to accommodate and receive an assembly 10 with housing 4 including the one or more image sensors (optical sensors) 1 (as shown in FIG. 1A) that is entirely removably from the cannula 100. Embodiments of the device 1000 may feature an opening 250 of the configurable guide insert 120 that may accommodate and correspond to any shape of the assembly 10 with housing 4 including the one or more image sensors (optical sensors) 1, so that the shape of the opening 250—whether orthogonal, cylindrical, or otherwise—may accommodate and correspond to a shape of a horizontal cross-section of the assembly 10 housing 4, so that the opening 250 of the configurable guide insert 120 may slidably receive an assembly 10 with housing 4 including the one or more image sensors (optical sensors) 1, as shown in FIG. 8A, for example.

In general, the opening 250 and slot opening 240 of the configurable guide insert 120 may be oriented for a patient's left side or right side, as noted by the markings 210 on the top portion 220 of the configurable guide insert 120. For example, the instrument may be oriented for an incision point at the ulnar portion of a patient's left hand or right hand. The left and right orientations of the configurable guide insert 120 enable the physician to ensure that the surgical implement that is inserted within the slot opening 240 of the configurable guide insert 120 will remain away from adjacent soft tissue structures during a surgical procedure. The configurable guide insert of FIG. 2A is shown in an orientation for a procedure in the left hand of a patient.

Referring to FIG. 2B showing the tubular body portion 126 of the cannula 100 with generic opening 260 of the base member 200, the base member 200 may feature the corresponding receiving points 270 to accept and engagingly lock to the corresponding engaging members 230 of the configurable guide insert 120. As aforementioned, the receiving points 270 may include ridges 271 that couple to the corresponding ridges 231 of the engaging members 230 of the configurable guide insert 120. The upper portion 115 of the base member 200 separates the generic opening 260 from the opening defined by the bore 116 between the axially-extending edges 121 of the sidewall 119.

Referring to FIG. 2C showing the obturator 118 of the device 1000, the proximal end 290 of the obturator 118 may feature markings 280 that assist the physician in orienting the obturator 118 according to whether the device 1000 will be inserted into an incision point in either the left side or right side of a patient. The markings 280 may comprise letters (“L” or “R”) or words (“LEFT” or “RIGHT”) or may alternatively comprise anatomic references, such as “ULNAR”, “RADIAL”, “VOLAR” or “DORSAL”. As such, the obturator 118 may interface with the device 1000 and configurable guide insert 120 that is oriented for a patient's left hand or right hand, for example. The obturator 118 may be placed within the tubular body portion 126 of the cannula 100 by inserting the obturator 118 through the opening 250 of the configurable guide insert 120 and through the generic opening 260 of the base member 200. As described previously, the obturator 118 prevents the entry of the soft tissue structures within the bore 116 of the tubular body 126 when the surgeon initially inserts the cannula 100 within an incision point of the tissue during a procedure.

Referring to FIG. 2D, a view of the surgical device 1000 is provided to illustrate the device 1000 with the configurable guide insert 120 and the obturator 118 in place within the cannula 100. As will be described in further detail, the configurable guide insert 120 may be positioned in one of two orientations. The configurable guide insert 120 may be inserted into and rigidly attached to the cannula 100 at an engagement point adjacent to the generic opening of the base member 200. The obturator 118 provides for rigidity during the initial insertion of the device 1000 into the surgical incision and also prevents other soft tissue anatomy from entering into the bore 116 between the axially-extending edges 121 of the sidewall 119.

Referring to FIG. 3A, a view of a configurable guide insert 120 that is fixed within a cannula 100 is shown. The configuration of FIG. 3A may be utilized with embodiments of the surgical device 1000 with an assembly 10 including the one or more image sensors (optical sensors) 1 that is entirely removably from the cannula 100. FIG. 3A illustrates the configurable guide insert 120 lockingly engaged within the base member 200 of the cannula 100. As shown in FIG. 3A, the corresponding receiving points 270 of the base member 200 accept and engagingly lock to the engaging members 230 of the configurable guide insert 120. In particular, the receiving points 270 may include ridges 271 that couple to the corresponding ridges 231 of the engaging members 230 of the configurable guide insert 120. Further, the base member 200 with configurable guide insert 120 in place within the base member 200 defines a combination track 255. The combination track 255 of the configurable guide insert 120 may include one contiguous opening with space to accommodate insertion of an assembly 10 including the one or more image sensors 1 (as shown in FIG. 1A) and a surgical instrument 300, such as a cutting implement or rasp, where the assembly 10 and surgical instrument 300 are restricted to only stay within their respective designated regions within the singular contiguous opening of the combination track 255 of the configurable guide insert 120. More specifically, the singular contiguous opening through the combination track 255 of the configurable guide insert 120 may include at least the opening 250 and the slot opening 240, which define axially-extending spaces for receiving other components of the surgical device 1000. The opening 250 and slot opening 240 define the designated regions within the singular contiguous opening through the combination track 255 of the configurable guide insert 120 for the restricted positioning and movement of the assembly 10 and surgical instrument 300 within the combination track 255 of the configurable guide insert 120. For example, the opening 250 may receive an assembly 10 including the one or more image sensors (optical sensors) 1 (as shown in FIG. 1A) that is entirely removably from the cannula 100. The slot opening 240 may receive a surgical implement 300, such as a cutting tool or scalpel. The opening 250 within the configurable guide insert 120 extends through the generic opening 260 (shown in FIG. 2B) of the base member 200 into a bore 116 for slidably receiving the assembly 10 including the image sensor 1, housing 4, and additional optical imaging components that are connected to the housing 4, such as the flexible circuitry 3 and the rigid or semi-rigid conduit 5 for enclosing the flexible circuitry 3. The slot opening 240 within the configurable guide insert 120 extends through the generic opening 260 (shown in FIG. 2B) of the base member 200 into an action area outside of and adjacent to the bore 116 for slidably receiving a surgical instrument 300, such as a cutting implement or rasp, for example. As used herein, an area is “outside of and adjacent to” the bore 116 when a corresponding space is non-axial with the bore 116 and adjacent to the region defined by the bore 116.

While specific geometries and configurations are illustrated in the drawings for the opening 250 and a slot opening 240, one of ordinary skill in the art would understand based on this disclosure that any complementary shaped interface may be respectively provided between the opening 250 and a slot opening 240 of the configurable guide insert 120 and the assembly 10 and surgical instrument 300. For example, it is understood that the opening 250, as shown in FIG. 3A, may feature a different shape, such as the rectangular shape of the slot 400 for receiving the image sensor 1 and housing 4, as shown in FIG. 1A.

Referring again to FIG. 3A, as will be described in more detail, the slot opening 240 and surgical implement 300 (as shown in FIGS. 1A-1B) may include mating features to couple the surgical implement to the slot opening 240 of the base member 200, which allows for guided sliding of the surgical implement through the configurable guide insert 120 and generic opening 260 (shown in FIG. 2B) of the base member 200 into an action area adjacent to the bore 116. The surgical implement 300 may be anyone one of the tissue manipulation devices 810, 820, 830 shown in FIGS. 8B-8D, which may be one of a rasp 810, reverse cutting knife 820, or forward cutting knife 830. The surgical device 1000 may provide the advantage of positioning the surgical implement 300 above and laterally adjacent to the assembly 10 including the one or more image sensors (optical sensors) 1 that is entirely removably from the cannula 100. In particular, the surgical device 1000 offers optimum visibility via the image sensors 1 of the assembly 10 for the physician to view the surgical implement 300 during a surgical procedure. Further, the surgical device 1000 may also simplify insertion of the cannula 100 at a narrow incision point since the assembly 10 including the image sensors 1 and surgical implement 300 may be positioned laterally adjacent to each other, rather than in a top-to-bottom arrangement. An orientation and positional relationship between the assembly 10 and surgical instrument 300 may be determined based upon a configuration of the configurable guide insert 120 in terms of whether the configurable guide insert 120 features the openings 250, 240 in a side-to-side configuration or a top-top-bottom configuration. As understood by those of skill in the art, although the configurable guide insert 120 as shown in the embodiment of FIG. 3A features a side-to-side configuration of the openings 250, 240, additional configurations of the configurable guide insert 120 may be contemplated featuring a top-to-bottom configuration of the openings 250, 240.

Referring again to FIG. 3A, the assembly 10 with image sensors 1 and surgical implement 300 may be slidably inserted within the opening 250 and slot opening 240, respectively, of the combination track 255. The opening 250 and slot opening 240 each creates an opening so that the assembly 10 with image sensors 1 and surgical implement 300 may be respectively introduced through the opening 250 and slot opening 240 along an axis parallel to the axis of the bore 116. The opening 250 and slot opening 240 may be connected openings, forming an inlet through the generic opening 260 of the base member 200 to an area outside of and adjacent to the bore 116, which may be an area radially between the axially-extending edges 121 of the sidewall 119. As used herein, an area is “radially between” the edges 121 when a corresponding space is between parallel planes which pass through the edges 121.

Referring again to FIG. 3A, the configurable guide insert 120 may include a top portion 220 with markings 210 to assist the user in selecting an orientation of the configurable guide insert 120 within the base member 200 of the cannula 100, for a surgical procedure in either the left or right side of a patient.

FIG. 3B provides another embodiment of the device 1000 showing a perspective view of a permanently coupled, positionable configurable guide insert 320 that is wholly contained by and attached to the base member 200 of the cannula 100. The permanently coupled, positionable configurable guide insert 320 of the embodiment in FIG. 3B is permanently coupled to the cannula 100, yet a user may rotate the configurable guide insert 320 180 degrees into place for each of the right and left orientations of the device, as indicated by the arrows in FIG. 3B. Although the permanently coupled, positionable configurable guide insert 320 shown in FIG. 3B features a hexagonal shape to accommodate the rotational functionality, one of ordinary skill in the art would understand based on this disclosure that any complementary shaped interface can be provided between the permanently coupled, positionable configurable guide insert 320 and the base member 200 of the cannula 100 to achieve the functionality to rotate the permanently coupled, positionable configurable guide insert 320 180 degrees into either the left or right orientation of the device. It is further understood that known mechanisms may be implemented to accommodate the permanent coupling of the configurable guide insert 320 to the base member 200 while also facilitating the user's ability to rotate the configurable guide insert 320 180 degrees into either the left or right orientation of the device. For example, the configurable guide insert 320 of the embodiment of FIG. 3B may be positionable for each of the left or right orientations of the device by operation of a push button (not pictured), such that a user may operate the push button or other mechanism to rotate the configurable guide insert 320 180 degrees within the base member 200 of the cannula 100, with the configurable guide insert 320 being permanently coupled to the base member 200. Of course, although the embodiment of FIG. 3B is shown in a left orientation, for a surgical procedure in a left side of a patient, a user may quickly rotate the configurable guide insert 320 into the alternative right orientation, for a surgical procedure in a right side of a patient.

Referring again to FIG. 3B in conjunction with FIG. 3A, although not shown in FIG. 3B, it is understood that the embodiment of FIG. 3B with permanently coupled, positionable configurable guide insert 320 further includes the combination track 255 with the opening 250 and the slot opening 240, as previously described in relation to FIG. 3A. The configurable guide insert 320 in the embodiment of FIG. 3B may include one contiguous opening with space to accommodate insertion of multiple surgical instruments, where the surgical instruments are restricted to only stay within their respective designated regions within the singular contiguous opening of the combination track 255 of the configurable guide insert 320. More specifically, the singular contiguous opening through the combination track 255 of the configurable guide insert 320 may include at least the opening 250 and the slot opening 240, which define axially-extending spaces for receiving other components of the surgical device. The opening 250 and slot opening 240 define the designated regions within the singular contiguous opening through the combination track 255 of the configurable guide insert 320 for the restricted positioning and movement of the surgical instruments within the combination track 255 of the configurable guide insert 320. For example, the opening 250 may receive an assembly 10 including the one or more image sensors (optical sensors) 1 (as shown in FIG. 1A) that is entirely removably from the cannula 100. The slot opening 240 may receive a surgical implement 300, such as a cutting tool or scalpel. Notably, when a user rotates the configurable guide insert 320 of the embodiment of FIG. 3B 180 degrees into either the left or right orientation, the contiguous opening of the configurable guide insert 320 with opening 250 and slot opening 240 is axially aligned with the generic opening 260 (shown in FIG. 2B) of the base member 200 to slidably receive surgical implements into the bore 116. When a user rotates the permanently coupled, positionable configurable guide insert 320 of FIG. 3B into either the left or right orientation, the opening 250 may be aligned upon rotation to extend axially through the generic opening 260 (shown in FIG. 2B) of the base member 200 into the bore 116 to slidably receive an assembly 10 including the one or more image sensors (optical sensors) 1 (as shown in FIG. 1A), and the slot opening 240 may be aligned to extend axially through the generic opening 260 (shown in FIG. 2B) of the base member 200 into an action area outside of and adjacent to the bore 116 to slidably receive a surgical implement 300. As used herein, an area is “outside of and adjacent to” the bore 116 when a corresponding space is non-axial with the bore 116 and adjacent to the region defined by the bore 116.

Referring again to FIG. 3B, it is further understood that the slot opening 240 and surgical implement 300 may include mating features to couple the surgical implement 300 to the slot opening 240 of the configurable guide insert 320, which allows for guided sliding of the surgical implement 300 through the configurable guide insert 320 and generic opening 260 (shown in FIG. 2B) of the base member 200 into an action area adjacent to the bore 116. A first surgical implement may be an assembly 10 including the one or more image sensors (optical sensors) 1 (as shown in FIG. 1A), and a second surgical implement may be anyone one of the tissue manipulation devices 810, 820, 830 shown in FIGS. 8B-8D, which may be one of a rasp 810, reverse cutting knife 820, or forward cutting knife 830. The surgical device as shown in FIG. 3B may provide the advantage of positioning a surgical implement 300 (that may be a second surgical implement) laterally adjacent to an assembly 10 including the one or more image sensors (optical sensors) 1 (as shown in FIG. 1A). In particular, the surgical device 1000 offers optimum visibility via an assembly 10 including the one or more image sensors (optical sensors) 1 for the physician to view the surgical implement 300 during a surgical procedure. Further, the surgical device 1000 also simplifies insertion of the cannula 100 at a narrow incision point since the assembly 10 including the image sensors 1 and surgical implement 300 may be positioned laterally adjacent to each other, rather than in a top-to-bottom arrangement. An orientation and positional relationship between the assembly 10 and surgical instrument 300 may be determined based upon a configuration of the configurable guide insert 320 in terms of whether the configurable guide insert 320 features the openings 250, 240 in a side-to-side configuration or a top-top-bottom configuration. As understood by those of skill in the art, although a configurable guide insert 320 as shown in the embodiment of FIG. 3A may feature a side-to-side configuration of the openings 250, 240, additional configurations of the configurable guide insert 320 may be contemplated featuring a top-to-bottom configuration of the openings 250, 240.

Referring again to FIG. 3B, it is understood that an assembly 10 including the image sensors 1 and surgical implement 300 may be slidably inserted within the opening 250 and slot opening 240, respectively, of the combination track 255. The opening 250 and slot opening 240 each create an opening so that an assembly 10 including the image sensors 1 and surgical implement 300 may be respectively introduced through the opening 250 and slot opening 240 along an axis parallel to the axis of the bore 116. The opening 250 and slot opening 240 may be connected openings, forming an inlet through the generic opening 260 of the base member 200 to an area outside of and adjacent to the bore 116, which may be an area radially between the axially-extending edges 121 of the sidewall 119. As used herein, an area is “radially between” the edges 121 when a corresponding space is between parallel planes which pass through the edges 121. Referring again to FIG. 3B, the permanently coupled, positionable configurable guide insert 320 may further include a top portion 220 with markings 210 to assist the user in selecting an orientation of the permanently coupled, positionable configurable guide insert 320 within the base member 200 of the cannula 100, for a surgical procedure in either the left or right side of a patient.

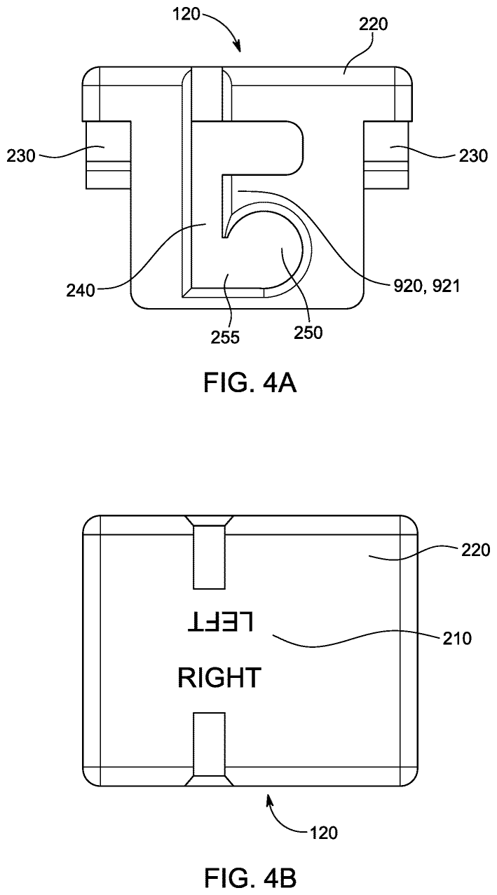

Referring to FIGS. 4A-4B, the configurable guide insert 120 in the disclosed embodiments may detachably couple to the base member 200 of the cannula 100. FIGS. 4A-4B provide expanded views of a configurable guide insert 120 shown in an orientation for a procedure in the right side of a patient (e.g., the right hand), the slot opening 240 and opening 250 of the configurable guide insert 120 may be oriented for a patient's right hand, as noted by the marking 210 on the top portion 220 of the configurable guide insert 120. As shown in the right orientation in FIGS. 4A-4B, the slot opening 240 may be positioned along the ulnar side of the patient's right wrist, so that the surgical implement 300 (as shown in FIG. 1A), such as a cutting instrument, that is inserted within the slot opening 240 of the configurable guide insert 120 will remain away from adjacent soft tissue structures of the right hand during a surgical procedure. Further, the expanded view provided by FIG. 4A for the configurable guide insert 120 also shows the engaging members 230 along with the combination track 255 having the opening 250 and slot opening 240. As will be described in further detail, the combination track 255 of the configurable guide insert 120 may also include the mating feature 920 including the protuberance 921 that matingly couples to the corresponding mating features of the surgical implement 300, such as a cutting implement with a blade, for example.

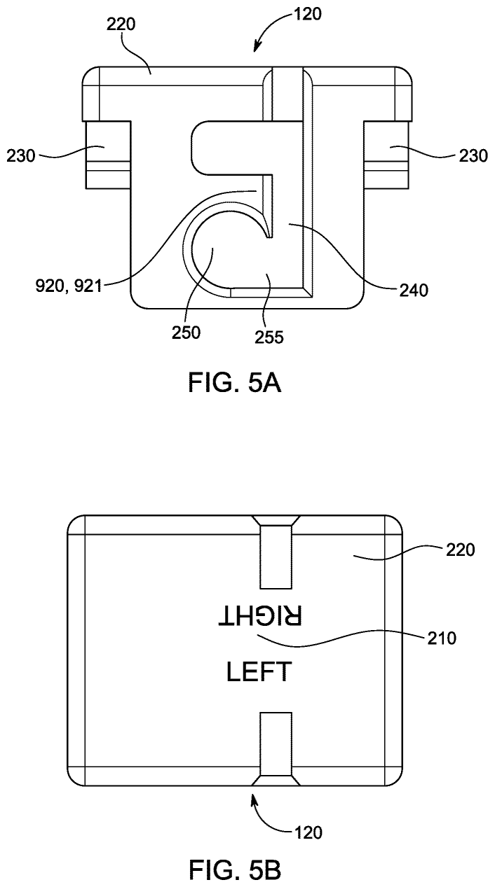

Similarly, FIGS. 5A-5B provide expanded views of the configurable guide insert 120 shown in an orientation for a procedure in the left side of a patient (e.g., the left hand), the slot opening 240 and opening 250 of the configurable guide insert 120 may be oriented for a patient's left hand, as noted by the markings 210 on the top portion 220 of the configurable guide insert 120. As shown in the left orientation in FIGS. 5A-5B, the slot opening 240 may be positioned along the ulnar side of the patient's left wrist, so that the surgical implement 300 (as shown in FIG. 1A), such as a cutting instrument, that is inserted within the slot opening 240 of the configurable guide insert 120 will remain away from adjacent soft tissue structures of the left hand during a surgical procedure. Further, the expanded view provided by FIG. 5A for the configurable guide insert 120 also shows the engaging members 230 along with the combination track 255 having the opening 250 and slot opening 240. As will be described in further detail, the combination track 255 of the configurable guide insert 120 may also include the mating feature 920 including the protuberance 921 that matingly couples to the corresponding mating features of the second surgical implement, such as a cutting implement with a blade, for example.

FIGS. 6A-6D provide additional views of the configurable guide insert 120 in a left orientation within the base member 200 of the cannula 100. When the configurable guide insert 120 is lockingly engaged within the base member 200 in the left orientation, the markings 210 on the top portion 220 of the configurable guide insert 120 may indicate a letter (“L”) or word (“LEFT”) or an anatomic reference, such as “ULNAR”, “RADIAL”, “VOLAR” or “DORSAL”. In particular, the markings 210 on the top portion 220 of the configurable guide insert 120 indicate the correct orientation of the device 1000 for the left orientation when the device 1000 is viewed from the proximal end 112 of the device 1000. For example, as shown in FIG. 6C, when viewing the device 1000 from the proximal end 112, the markings 210 on the top portion 220 of the configurable guide insert 120 indicate “LEFT” so that the physician knows that the device 1000 will be inserted into an incision point 610 in the ulnar side of a patient's left hand 600, as shown in FIG. 6D. In the left orientation shown in FIGS. 6A-6C, the surgical implement 300 (as shown in FIG. 1A), such as a cutting instrument that is inserted within the slot opening 240 of the configurable guide insert 120, will remain away from adjacent soft tissue structures of the left hand 600 during a surgical procedure. Although FIG. 6D provides an illustration of a patient's left hand, it is understood that the device 1000 may be utilized for surgical procedures at incision points within additional anatomical locations of the body, such as a left foot, for example.

Similarly, FIGS. 7A-7D provide additional views of the configurable guide insert 120 in a right orientation within the base member 200 of the cannula 100. When the configurable guide insert 120 is lockingly engaged within the base member 200 in the right orientation, the markings 210 on the top portion 220 of the configurable guide insert 120 may indicate a letter (“R”) or word (“RIGHT”) or an anatomic reference, such as “ULNAR”, “RADIAL”, “VOLAR” or “DORSAL”. In particular, the markings 210 on the top portion 220 of the configurable guide insert 120 indicate the correct orientation of the device 1000 for the right orientation when the device 1000 is viewed from the proximal end 112 of the device 1000. For example, as shown in FIG. 7C, when viewing the device 1000 from the proximal end 112, the markings 210 on the top portion 220 of the configurable guide insert 120 indicate “RIGHT” so that the physician knows that the device 1000 will be inserted into an incision point 710 in the ulnar side of a patient's right hand 700, as shown in FIG. 7D. In the right orientation shown in FIGS. 7A-7C, the surgical implement 300 (as shown in FIG. 1A), such as a cutting instrument, that is inserted within the slot opening 240 of the configurable guide insert 120, will remain away from adjacent soft tissue structures of the right hand 700 during a surgical procedure. Although FIG. 7D provides an illustration of a patient's right hand, it is understood that the device 1000 may be utilized for surgical procedures at incision points within additional anatomical locations of the body, such as a right foot, for example.

Based on the shape of the obturator 118 (shown in FIG. 2C) relative to the shapes of the opening 250 and slot opening 240 of the configurable guide insert 120 (as shown in FIG. 3A), it is not possible to insert the device 1000 with obturator 118 in the “right” configuration for a surgical procedure in the right hand of a patient while the configurable guide insert 120 is oriented for the “left” configuration, as shown in FIGS. 6A-6C. Similarly, it is not possible to insert the device 1000 with obturator 118 in the “left” configuration for a surgical procedure in the left hand of a patient while the configurable guide insert 120 is oriented for the “right” configuration, as shown in FIGS. 7A-7C. The obturator 118 must be inserted in the same configuration (i.e., left or right) as the configurable guide insert 120 based upon the shape of the obturator 118 in relation to the shapes of the opening 250 and slot opening 240 of the configurable guide insert 120

As aforementioned, the configurable guide insert 120 may be detachably coupled to the base member 200 of the cannula 100 in a left or right orientation with respect to a patient's left or right hand, for example. As used herein, the term detachably coupled means that the configurable guide insert 120 is rigidly attached to the base member 200 of the cannula 100 so that it can accommodate tools for surgical procedures in either a patient's left or right side, depending on whether the configurable guide insert 120 is detachably coupled to the base member 200 in a left or right orientation. Markings 210 on the configurable guide insert 120 indicate the orientation of the device 1000, so that the physician may quickly assemble the device 1000 by detachably coupling the configurable guide insert 120 to the base member 200 of the cannula 100, for a rigid attachment of the cannula 100, configurable guide insert 120 and obturator 118, in the selected orientation, for insertion of the device 1000 at a designated incision point in a left side or right side of a patient. When detachably coupling the configurable guide insert 120 in either the “left” orientation or the “right” orientation, the configurable guide insert 120 may be rotated 180 degrees relative the axis defined by the cannula 100 and bore 116. When the configurable guide insert 120 is detachably coupled to the base member 200 in either the left or right orientation, an imaginary axis extends through the opening 250 of the configurable guide insert 120 and into the bore 116, and another imaginary axis extends through the slot opening 240 of the configurable guide insert 120 and into the bore 116. The configurable guide insert 120 may be quickly rotated at the time of the surgical procedure to detachably couple the configurable guide insert 120 to the base member 200 of the cannula 100 in either the left or right orientation for assembly of the device 1000.

In general, each of the assembly 10 and the surgical instrument 300 may be slidably received in the combination track 255 of the configurable guide insert 120, when the configurable guide insert 120 is lockingly engaged within the base member 200 of the surgical device 1000. FIGS. 8A-8D illustrate embodiments of an assembly 10 and tissue manipulation devices 810, 820, 830. In one embodiment, the tissue manipulation devices 810, 820, 830 may be one of a rasp 810, reverse cutting knife 820, or forward cutting knife 830, as shown in FIGS. 8A-8D. When the tissue manipulation devices 810, 820, 830 are used in conjunction with the surgical device 1000, the surgical implement 300 (as shown in FIG. 1A) may be any of the tissue manipulation devices 810, 820, 830.

Referring again to FIGS. 8A-8D in conjunction with FIG. 3A, in use, the assembly 10 with image sensors 1 slides through the opening 250 of the combination track 255, into the bore 116 until it reaches the distal end 114 (shown in FIGS. 1A-1B) of the device 1000. The assembly 10 with image sensors 1 thereby supplies a visualization of a target tissue at which the distal end 114 is positioned in a patient. The opening of the bore 116 at the distal end 114 provides an axial line of sight for the assembly 10 with image sensors 1 while the opening formed by the sidewall 119 provides a radial line of sight. The surgical implement 300 (tissue manipulation device) slides through the slot opening 240 of the combination track 255, adjacent to the assembly 10 with image sensors 1. The slot opening 240 guides the surgical implement 300 such that the surgical procedure may be carried out with assistance from the visualization provided by the assembly 10 with image sensors 1. As seen in FIGS. 8A-8D, the tissue manipulation devices 810, 820, 830 may include various features, including, for example, acting features 811, 821, 831, sliding features, and handle features 851, 852, 853. The acting features 811, 821, 831 are components (e.g., blade, clamp, hook, etc.) which are capable of completing a surgical task (e.g., cutting, moving, modifying, etc.). The sliding features include a structure which interacts with the cannula 100 to allow the tissue manipulation devices 810, 820, 830 to be guided relative to the assembly 10 with image sensors 1. The handle features 851, 852, 853 are, for example, upwardly-extending handles which allow a physician to easily grasp and move the tissue manipulation devices 810, 820, 830. Focusing now on the interplay between the assembly 10 with image sensors 1, tissue manipulation devices 810, 820, 830 (as shown in FIGS. 8A-8D), and the combination track 255 (as shown in FIGS. 5A and 6A), in some embodiments, such as those of FIGS. 5A and 6A, the combination track 255 includes a mating feature 920 including a protuberance 921, which couples to the corresponding mating feature of the surgical implement 300, such as the groove 310, as shown in FIG. 1A. As aforementioned in reference to FIGS. 8A-8D, the surgical implement 300 may be any of the tissue manipulation devices 810, 820, 830.

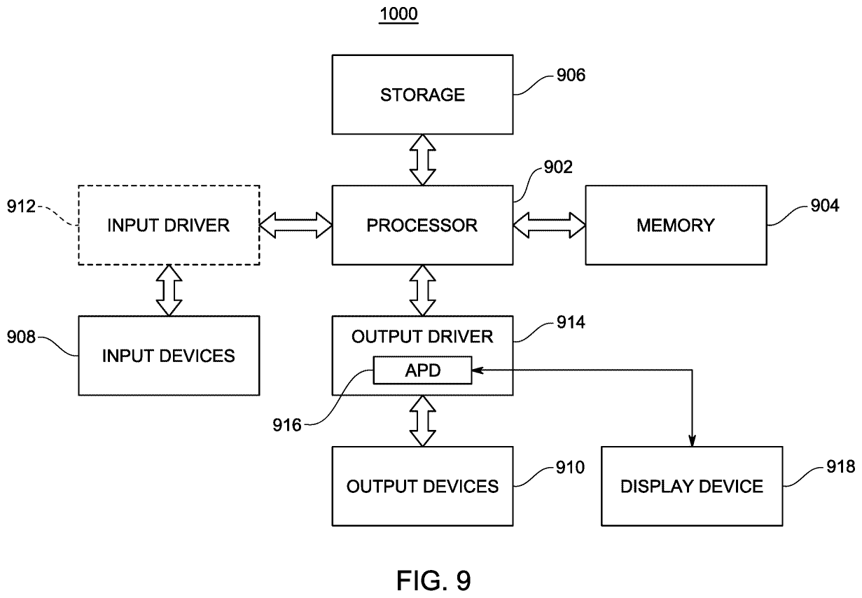

FIG. 9 is a block diagram of a surgical device 1000 in which one or more disclosed embodiments can be implemented. The device 1000 may include an assembly 10 with at least one image sensor 1, where the assembly 10 may be permanently fixed and integral to the cannula 100 (as shown in FIG. 1B for example). In other embodiments, the device 1000 may include an assembly 10 with at least one image sensor 1, where the assembly 10 is entirely removable from the cannula 100 (as shown in FIG. 1A). In other embodiments, the device 1000 may include an assembly 10 with at least one image sensor 1, where the assembly 10 for at least one image sensor 1 may be permanently fixed to a lower portion of the cannula 100 while also having additional capability for slidable movement and adjustable positioning within the cannula 100 along a longitudinal axis of the cannula 100.

As aforementioned, the assembly 10 of the device 1000 may include additional optical components including a light source 2 such as an LED, a flexible circuitry 3 for carrying the captured image(s) from the one or more image sensors 1 to a coupler 6, an optical grade protective housing 4 covering the one or more image sensors 1 and the light source 2, a rigid or semi-rigid conduit 5 for enclosing the flexible circuitry 3, a coupler 6 for connection to an optical display, and a connection mechanism 7 for optical image transfer to another device. It is understood that the flexible circuitry 3 for carrying the captured image(s) from the one or more image sensors 1 to a coupler 6 for connection to an optical display, and the connection mechanism 7 for optical image transfer to another device, may enable the device 1000 to communicatively couple with additional components including at least a processor, a memory, a storage, one or more input devices, and one or more output devices. When deploying a cannula 100 with multiple sensors 1, image data from each of the sensors 1 may be transmitted to a single display screen and viewed by the physician at the same time in a multi-view display.

Referring again to FIG. 9, the device 1000 may be communicatively coupled to a processor 902, a memory 904, a storage 906, one or more input devices 908, and one or more output devices 910. The device 1000 may also optionally be communicatively coupled to an input driver 912 and an output driver 914. It is understood that the device 1000 may include additional components not shown in FIG. 9.

The processor 902 can include a central processing unit (CPU), a graphics processing unit (GPU), a CPU and GPU located on the same die, or one or more processor cores, wherein each processor core can be a CPU or a GPU. The memory 904 can be located on the same die as the processor 902, or can be located separately from the processor 902. The memory 904 can include a volatile or non-volatile memory, for example, random access memory (RAM), dynamic RAM, or a cache.

The storage 906 may include a fixed or removable storage, for example. The input devices 908 can include a keyboard, a keypad, a touch screen, a touch pad, a detector, a microphone, one or more image sensors 1 as described herein, an accelerometer, a gyroscope, a biometric scanner, or a network connection (e.g., a wireless local area network card for transmission and/or reception of wireless IEEE 802 signals). The output devices 910 can include a display, a speaker, a printer, a haptic feedback device, one or more lights, an antenna, or a network connection (e.g., a wireless local area network card for transmission and/or reception of wireless IEEE 802 signals).

The input driver 912 may communicate with the processor 902 and the input devices 908, and may permit the processor 902 to receive input from the input devices 908. The output driver 914 may communicate with the processor 902 and the output devices 910, and may permit the processor 902 to send output to the output devices 910. It is noted that the input driver 912 and the output driver 914 are optional components, and that the device 1000 may operate in the same manner if the input driver 912 and the output driver 914 are not present. The output driver 914 may include an accelerated processing device (“APD”) 916 which may be coupled to a display device 918. The APD may be configured to accept compute commands and graphics rendering commands from processor 902, to process those compute and graphics rendering commands, and to provide pixel output to display device 918 for display. As described in further detail below, the APD 916 may include one or more parallel processing units configured to perform computations in accordance with a single-instruction-multiple-data (“SIMD”) paradigm. Thus, although various functionality is described herein as being performed by or in conjunction with the APD 916, the functionality described as being performed by the APD 916 may also be performed by other computing devices having similar capabilities that are not driven by a host processor (e.g., processor 902) and configured to provide graphical output to a display device 918. For example, it is contemplated that any processing system that performs processing tasks in accordance with a SIMD paradigm may be configured to perform the functionality described herein. Alternatively, it is contemplated that computing systems that do not perform processing tasks in accordance with a SIMD paradigm may perform the functionality described herein.

The presently disclosed embodiments may provide a more accurate and repeatedly consistent method of visualization and modification of soft tissue structures. The disclosed embodiments may prescribe a predefined motion for tissue manipulation devices, which helps to ensure the appropriate soft tissue structure is manipulated by the appropriate amount, and helps to prevent unwanted manipulation of adjacent soft tissue structures. The disclosed embodiments may prevent excessive or inadvertent motion of tissue manipulation devices by guiding movement thereof. The disclosed embodiments may also allow independent motion of implements relative to one or more image sensors during a surgical procedure, while maintaining close proximity between the implements and the one or more image sensors for enhanced visualization of surgical maneuvers. The disclosed surgical device facilitates simplified surgical steps and may provide consistency as multiple types of implements may be passed through the cannula for use in conjunction with the one or more image sensors.

Having thus described the presently preferred embodiments in detail, it is to be appreciated and will be apparent to those skilled in the art that many physical changes, only a few of which are exemplified in the detailed description of the invention, could be made without altering the inventive concepts and principles embodied therein. It is also to be appreciated that numerous embodiments incorporating only part of the preferred embodiment are possible which do not alter, with respect to those parts, the inventive concepts and principles embodied therein. The present embodiments and optional configurations are therefore to be considered in all respects as exemplary and/or illustrative and not restrictive, of the scope of the invention being indicated by the appended claims rather than by the foregoing description, and all alternate embodiments and changes to this embodiment which come within the meaning and range of equivalency of said claims are therefore to be embraced therein.

1. A cannula for use as part of a surgical device, comprising:
at least one embedded sensor;
an assembly within the cannula including the at least one embedded sensor;
a housing member sealing the assembly and having an upper surface extending axially along a length of the cannula,
the at least one embedded sensor disposed within the upper surface of the housing member;
a processor and a memory communicatively coupled to the at least one embedded sensor;
a base member at a proximal end of the cannula;
a sidewall connected to the base member and extending in an axial direction from the proximal end to a distal end of the cannula;
a bore extending in the axial direction defined by each of the upper surface of the housing member and the base member and the sidewall; and
an opening extending axially through the base member and into the bore between axially-extending edges of the sidewall,
wherein the base member is configured to slidably receive at least one surgical implement through the opening and into the bore, and
wherein the at least one embedded sensor detects an image data of a target tissue and an adjacent tissue, the target tissue to be manipulated by the at least one surgical implement.

2. The cannula of claim 1, wherein the at least one embedded sensor is an image sensor configured to transmit the image data to a display, and
the image sensor comprises an adjacent region in the upper surface for mounting one or more embedded LEDs within the housing member, each of the embedded LEDs flush with the upper surface.

3. The cannula of claim 1, wherein the at least one embedded sensor comprises a plurality of image sensors configured to transmit a respective image data to a display for visualization of the respective image data of each of the plurality of image sensors, and
each of the plurality of image sensors comprises an adjacent region in the upper surface for mounting one or more embedded LEDs within the housing member, each of the embedded LEDs flush with the upper surface.

4. The cannula of claim 3, wherein the housing member including the plurality of image sensors and the one or more embedded LEDs comprises an imaging probe, the imaging probe configured to slide along the length of the cannula between the axially-extending edges of the sidewall in the axial direction between the proximal end and the distal end of the cannula.

5. The cannula of claim 4, wherein the base member is configured to slidably receive the imaging probe through the opening and between the axially-extending edges of the sidewall.

6. The cannula of claim 5, the imaging probe further comprising at least one protrusion and the cannula further comprising at least one fixed position engagement point comprising a recess,
the at least one protrusion configured to matingly couple with the at least one fixed position engagement point,
wherein the at least one protrusion that is configured to matingly couple with the at least one fixed position engagement point provides a selectable incremental positioning of the imaging probe within the cannula.

7. The cannula of claim 6, wherein the base member includes a configurable guide insert detachably coupled to the base member, the configurable guide insert including a first opening extending axially through the base member into an action area outside the opening into the bore and radially between edges of the sidewall, and
the configurable guide insert further including a second opening adjacent to
the first opening, the
second opening extending axially through the base member into the bore, the base member configured to slidably receive the housing member through the second opening and into the bore,
wherein the configurable guide insert is detachably coupled to be positionable in a configurable orientation comprising a right orientation for positioning of the at least one surgical implement for a surgical procedure in a right side of a patient, or a left orientation for positioning of the at least one surgical implement for the surgical procedure in a left side of the patient, and
wherein the configurable guide insert is configured for rotation of 180 degrees about an axis defined in relation to the cannula when positioning the configurable guide insert on the base member in the right orientation or in the left orientation.

8. A tissue visualization system for use as part of a surgical device, comprising:
a cannula having a proximal end and a distal end;
at least one embedded sensor;
an assembly within the cannula including the at least one embedded sensor;
a housing member sealing the assembly and having an upper surface extending axially along a length of the cannula,
the at least one embedded sensor disposed within the upper surface of the housing member;
a processor and a memory communicatively coupled to the at least one embedded sensor;
a base member at the proximal end of the cannula;
a sidewall connected to the base member and extending in an axial direction from the proximal end to the distal end of the cannula;
a bore extending in the axial direction defined by each of the upper surface of the housing member and the base member and the sidewall; and
an opening extending axially through the base member and into the bore between axially-extending edges of the sidewall,
wherein the base member is configured to slidably receive at least one surgical implement through the opening and into the bore, and
wherein the at least one embedded sensor detects an image data of a target tissue and an adjacent tissue, the target tissue to be manipulated by the at least one surgical implement.

9. The tissue visualization system of claim 8, wherein the at least one embedded sensor is an image sensor configured to transmit the image data to a display, and
the image sensor comprises an adjacent region in the upper surface for mounting one or more embedded LEDs within the housing member, each of the embedded LEDs flush with the upper surface.

10. The tissue visualization system of claim 8, wherein the at least one embedded sensor comprises a plurality of image sensors configured to transmit a respective image data to a display for visualization of the respective image data of each of the plurality of image sensors, and
each of the plurality of image sensors comprises an adjacent region in the upper surface for mounting one or more embedded LEDs within the housing member, each of the embedded LEDs flush with the upper surface.

11. The tissue visualization system of claim 10, wherein the housing member including the plurality of image sensors and the one or more embedded LEDs comprises an imaging probe, the imaging probe configured to slide along the length of the cannula between the axially-extending edges of the sidewall in the axial direction between the proximal end and the distal end of the cannula.

12. The tissue visualization system of claim 11, wherein the base member is configured to slidably receive the imaging probe through the opening and between the axially-extending edges of the sidewall.

13. The tissue visualization system of claim 12, the imaging probe further comprising at least one protrusion and the cannula further comprising at least one fixed position engagement point comprising a recess,
the at least one protrusion configured to matingly couple with the at least one fixed position engagement point,
wherein the at least one protrusion that is configured to matingly couple with the at least one fixed position engagement point provides a selectable incremental positioning of the imaging probe within the cannula.

14. The tissue visualization system of claim 13, wherein the base member includes a configurable guide insert detachably coupled to the base member, the configurable guide insert including a first opening extending axially through the base member into an action area outside the opening into the bore and radially between edges of the sidewall, and
the configurable guide insert further including a second opening adjacent to
the first opening, the
second opening extending axially through the base member into the bore, the base member configured to slidably receive the housing member through the second opening and into the bore,
wherein the configurable guide insert is detachably coupled to be positionable in a configurable orientation comprising a right orientation for positioning of the at least one surgical implement for a surgical procedure in a right side of a patient, or a left orientation for positioning of the at least one surgical implement for the surgical procedure in a left side of the patient, and
wherein the configurable guide insert is configured for rotation of 180 degrees about an axis defined in relation to the cannula when positioning the configurable guide insert on the base member in the right orientation or in the left orientation.

15. A surgical device comprising:
a cannula having a proximal end and a distal end;
at least one embedded sensor;
an assembly within the cannula including the at least one embedded sensor;
a housing member sealing the assembly and having an upper surface extending axially along a length of the cannula,
the at least one embedded sensor disposed within the upper surface of the housing member;
a processor and a memory communicatively coupled to the at least one embedded sensor;
a base member at the proximal end of the cannula;
a sidewall connected to the base member and extending in an axial direction from the proximal end to the distal end of the cannula;
a bore extending in the axial direction defined by each of the upper surface of the housing member and the base member and the sidewall; and
an opening extending axially through the base member and into the bore between axially-extending edges of the sidewall,
wherein the base member is configured to slidably receive at least one surgical implement through the opening and into the bore, and
wherein the at least one embedded sensor detects an image data of a target tissue and an adjacent tissue, the target tissue to be manipulated by the at least one surgical implement.

16. The surgical device of claim 15, wherein the at least one embedded sensor is an image sensor configured to transmit the image data to a display, and
the image sensor comprises an adjacent region in the upper surface for mounting one or more embedded LEDs within the housing member, each of the embedded LEDs flush with the upper surface.

17. The surgical device of claim 15, wherein the at least one embedded sensor comprises a plurality of image sensors configured to transmit a respective image data to a display for visualization of the respective image data of each of the plurality of image sensors, and
each of the plurality of image sensors comprises an adjacent region in the upper surface for mounting one or more embedded LEDs within the housing member, each of the embedded LEDs flush with the upper surface.

18. The surgical device of claim 17, wherein the housing member including the plurality of image sensors and the one or more embedded LEDs comprises an imaging probe, the imaging probe configured to slide along the length of the cannula between the axially-extending edges of the sidewall in the axial direction between the proximal end and the distal end of the cannula.

19. The surgical device of claim 18, wherein the base member is configured to slidably receive the imaging probe through the opening and between the axially-extending edges of the sidewall.

20. The surgical device of claim 19, the imaging probe further comprising at least one protrusion and the cannula further comprising at least one fixed position engagement point comprising a recess,
the at least one protrusion configured to matingly couple with the at least one fixed position engagement point,
wherein the at least one protrusion that is configured to matingly couple with the at least one fixed position engagement point provides a selectable incremental positioning of the imaging probe within the cannula.

21. The cannula of claim 1, further comprising:
a channel having an internal surface, the assembly having an exterior surface, the exterior surface of the assembly disposed within the internal surface of the channel, and
a shape of the internal surface of the channel corresponding to a shape of the exterior surface of the assembly.

22. The cannula of claim 21, wherein the shape of the internal surface of the channel corresponding to the shape of the exterior surface of the assembly is rectangular or cylindrical, and
the assembly disposed along the length of the cannula between the axially-extending edges of the sidewall in the axial direction between the proximal end and the distal end of the cannula.

23. The tissue visualization system of claim 8, further comprising:
a channel within the cannula having an internal surface, the assembly having an exterior surface, the exterior surface of the assembly disposed within the internal surface of the channel, and
a shape of the internal surface of the channel corresponding to a shape of the exterior surface of the assembly.

24. The tissue visualization system of claim 23, wherein the shape of the internal surface of the channel corresponding to the shape of the exterior surface of the assembly is rectangular or cylindrical, and
the assembly disposed along the length of the cannula between the axially-extending edges of the sidewall in the axial direction between the proximal end and the distal end of the cannula.

25. The surgical device of claim 15, further comprising:
a channel within the cannula having an internal surface, the assembly having an exterior surface, the exterior surface of the assembly disposed within the internal surface of the channel, and
a shape of the internal surface of the channel corresponding to a shape of the exterior surface of the assembly.

26. The surgical device of claim 25, wherein the shape of the internal surface of the channel corresponding to the shape of the exterior surface of the assembly is rectangular or cylindrical, and
the assembly disposed along the length of the cannula between the axially-extending edges of the sidewall in the axial direction between the proximal end and the distal end of the cannula.
